(19) United States
(12) Patent Application Publication
(10) Pub. No.: US 2020/0368211 A1
(43) Pub. Date: Nov. 26, 2020

(54) INHIBITION OF GAMMA-GLUTAMYLTRANSFERASE AND GLUTATHIONE CATABOLISM TO ENHANCE THE EFFICACY OF NF-KB SIGNALLING PATHWAY INHIBITORS

(71) Applicant: UNIVERSITAET HEIDELBERG, Heidelberg (DE)

(72) Inventors: Aleksandar RADUJKOVIC, Mannheim (DE); Thomas LUFT, Heidelberg (DE)

(73) Assignee: UNIVERSITAET HEIDELBERG, Heidelberg (DE)

(21) Appl. No.: 16/613,514

(22) PCT Filed: May 15, 2018
(86) PCT No.: PCT/EP2018/062609

§ 371 Date: Nov. 14, 2019

(30) Foreign Application Priority Data
May 16, 2017 (EP) 17171283.9

(51) Int. Cl. A61K 31/433; A61K 31/42; A61K 31/275; A61K 31/44; A61K 31/45; A61P 35/00

(52) U.S. Cl. CPC A61K 31/433; A61K 31/42; A61P 35/00; A61K 31/44; A61K 31/45; A61K 31/275

(57) ABSTRACT

The present invention relates to compositions for the treatment of a malignant disease and in particular to compositions comprising an NF-κB inhibitor and a γ-glutamyltransferase (γ-GT) inhibitor and therapeutic uses thereof. The invention further relates to in vitro methods of testing putative anti-tumour agents for their potential to act as therapeutically effective anti-tumour agents in vivo. Embodiments of the invention have been particularly developed for bringing NF-κB inhibitors to the clinic such as to provide alternative therapeutic options in the treatment of malignant diseases and will be described hereinafter with reference to this application. However, it will be appreciated that the invention is not limited to this particular field of use.

### FIELD OF THE INVENTION

The present invention relates to compositions for the treatment of a malignant disease and in particular to compositions comprising an NF-κB inhibitor and a γ-glutamyltransferase (γ-GT) inhibitor and therapeutic uses thereof. The invention further relates to in vitro methods of testing putative anti-tumour agents for their potential to act as therapeutically effective anti-tumour agents in vivo. Embodiments of the invention have been particularly developed for bringing NF-κB inhibitors to the clinic such as to provide alternative therapeutic options in the treatment of malignant diseases and will be described hereinafter with reference to this application. However, it will be appreciated that the invention is not limited to this particular field of use.

### BACKGROUND OF THE INVENTION

Any discussion of the background art throughout the specification should in no way be considered as an admission that such art is widely known or forms part of common general knowledge in the field.

Cancer is a leading cause of death in industrialised countries (World Health Organization 2005). While earlier detection and increased treatment options have led to a decline in mortality rates in recent years, most cancers remain incurable. Importantly, the term “cancer” is only an umbrella term including many vastly different malignant diseases, each with distinct characteristics leading to distinct therapeutic requirements and treatment options. In malignant cells the progressive sequence of mutations and epigenetic alterations derail cellular control mechanisms thereby promoting the transformation of cancer progenitor cells, ultimately leading to the establishment of the six hallmarks of malignant cells: (1) self-sufficiency in proliferative growth signals, (2) insensitivity to growth inhibitory signals, (3) evasion of apoptosis, (4) acquisition of limitless replicative potential, (5) induction of angiogenesis, and (6) induction of invasion and metastasis.

Genomic instability is common, if not universal, in advanced tumours. Thus, one major challenge for cancer researchers is to distinguish cancer-causing mutations from irrelevant alterations linked to complex cancer genotypes (Baud & Karin 2009). In this regard, it is noteworthy that alterations in the p53 tumour suppressor gene (referred to below as “p53” or “TP53”) stand out as the most common alterations in many cancers, e.g. 96% in ovarian serous carcinoma, 54% in invasive breast carcinomas, 86% in small cell lung cancer, and 75% in pancreas cancer (Kim et al. 2015). However, not all genetic alterations are biologically equivalent. The majority of alterations involve p53 missense mutations that result in the production of mutant p53 proteins. Such mutant p53 proteins lack normal p53 function and may concomitantly gain novel functions (gain-of-function mutations; GOF)—often with deleterious effects and in some cases subsequently leading to tumours becoming dependent on, i.e. being addicted to, the mutant p53 (Vaughan et al. 2016). Further, many cancers are susceptible to certain chemotherapeutic and/or radiotherapeutic regimes at the outset of treatment, they may develop resistance, i.e. non-responsiveness, to the therapy and the prevalence of drug resistant cancers has been increasing. While cellular mechanisms such as drug efflux can diminish a drug's efficacy, the genomic instability of many tumour cells appears to lead to further, possibly secondary mutations, which may selectively favour tumour survival and growth upon selective pressure of drug exposure, or may reduce the cancer cells' sensitivity to radiation. In this regard, it is important to remember that when a chemotherapeutic agent is administered, in particular when treating solid tumours, it is often difficult to systemically deliver a dosage sufficient to achieve an effective dose at the site of the tumour without causing detrimental side-effects due to the high, non-specific cytotoxicity of chemotherapeutic agents. Accordingly, cancer cells surviving an initial round of chemotherapy may well include a population of cells already having a certain, low degree of resistance to the chemotherapeutic agent. This population will continue to mutate and may therefore potentially acquire a higher degree of chemotherapeutic resistance. In addition, each round of chemotherapy inadvertently enriches for treatment resistant cells leading to highly chemotherapeutic resistant tumours.

Accordingly, in order to effectively treat tumours that have acquired resistance against the conventional first-line radio- and/or chemotherapeutic treatments, alternative treatment options are needed. Preferably, treatment options are needed that preferentially target malignant cells over healthy cells.

Inhibition of NF-κB signalling pathways has long been considered a potential therapeutic approach to treat malignant diseases and has been presented as a potential alternative treatment option for addressing resistance against conventional radio- and chemotherapeutic regimes. In fact, in the roughly 30 years since discovery of NF-κB the study of NF-κB signalling has become an industry, complete with website (www.nf-kb.org) and tens of thousands of publications (Gilmore 2006). Already in 2006 at least 785 inhibitors of NF-κB signalling (“NF-κB inhibitors”) had been identified, many with promising cytostatic and/or cytotoxic efficacy in vitro (Gilmore & Herscovitch 2006). For example, Dietrich et al. showed in 2011 that an immunosuppressive molecule with NF-κB inhibitory effect (A771726; leflunomide) approved for use in the treatment of rheumatoid arthritis and psoriatic arthritis could induce apoptosis in clinically refractory chronic lymphatic leukaemia (CLL) cells (Dietrich et al. 2011). However, with the growing number of NF-κB inhibitors the unfortunate discrepancy between in vitro and in-vivo efficacy of NF-κB inhibitors has also become more pronounced as only a very small number of in vitro NF-κB inhibitors have shown therapeutic efficacy in vivo leading to regulatory approval. In fact, even for these a few approved NF-κB inhibitors it is unclear whether their clinical efficacy is solely (or even partially) dependent on their ability to inhibit NF-κB signalling (Basseres & Baldwin 2006; Baud & Karin 2009).

U.S. Pat. Nos. 8,741,937 and 9,540,337 describe methods of inhibiting γ-GT using γ-GT inhibitors (a) to enhance the efficacy of clinically-relevant chemotherapeutic agents (i.e. of anti-tumour agents effective on tumour cell lines in vitro as well as on human tumours in vivo), (b) to enhance the efficacy of radiation-based cancer therapy, and (c) to treat or prevent reversible airway obstructions such as asthma, chronic obstructive pulmonary disease (COPD), allergic reaction, respiratory tract infections or upper respiratory tract diseases. Neither U.S. Pat. No. 8,741,937 nor U.S. Pat. No. 9,540,337 mention NF-κB inhibitors.

As such, the challenge to increase or enhance the in vivo efficacy of NF-κB inhibitors such that they may serve as therapeutic anti-cancer agents, either as stand-alone therapies or as adjuvants to existing or new therapies, remains. Accordingly a need in the art exists for improved NF-κB inhibitor compositions for the treatment of patients suffering from malignant disease. Further, in particular in light of the vast number of in vitro NF-κB inhibitors available, and the significant costs associated with bringing a promising pre-clinical NF-κB inhibitor to the clinic, there is an urgent need in the art for improved methods of identifying the in vitro NF-κB inhibitors with a high potential to achieve therapeutic efficacy also in vivo. At the same time, given the highly personalised manifestations in individual patients, improved methods of identifying individual patients who are susceptible to treatment with a particular NF-κB inhibitor are also needed.

### SUMMARY OF THE INVENTION

It is an object of the present invention to overcome or ameliorate at least one of the disadvantages of the prior art, or to provide a useful alternative. In particular, it is an object of the present invention to provide improved NF-κB inhibitor compositions and to provide improved methods of identifying in vitro NF-κB inhibitors suitable as stand-alone or combinatorial anti-cancer agents as well as of identifying individual patients susceptible to treatment with NF-κB inhibitors.

As indicated above, Dietrich et al. showed that an immunosuppressive molecule with NF-κB inhibitory effect (A771726; leflunomide) approved for use in the treatment of rheumatoid arthritis and psoriatic arthritis could inhibit proliferation and induce apoptosis in clinically refractory chronic lymphatic leukaemia (CLL) cells. However, the inventors' observations that the anti-proliferative and pro-apoptotic effects of the NF-κB inhibitor leflunomide were lost when the NF-κB inhibitor was administered to CLL patients with TP53 mutations (see Reference Example 1 below and FIG. 1) ultimately led to the unexpected findings underlying the present invention.

Namely, the inventors surprisingly found that the in vitro inhibition of proliferation and induction of apoptosis mediated by a number of NF-κB inhibitors, including leflunomide, was strongly diminished in cell cultures supplemented with human serum but could be restored and/or enhanced by the inhibition of γ-glutamyltransferase and/or by the inhibition of the glutathione catabolism. As such, the inventors found that in the presence of human serum, and without additional intervention, the NF-κB signalling pathway escaped the inhibitory action of a proven NF-κB inhibitor.

Accordingly, in a first aspect, the present invention relates to a composition comprising an NF-κB inhibitor and a γ-glutamyltransferase inhibitor. Typically, the composition is for use in medical treatment and, preferably, for use in the treatment of a malignant disease.

In embodiments of this aspect, the invention therefore also relates to methods of medical treatment, such as the treatment of a malignant disease, comprising the administration of an NF-κB inhibitor and a γ-glutamyltransferase inhibitor, as well as to use of an NF-κB inhibitor and a γ-glutamyltransferase inhibitor in the manufacture of a medicament for treating a malignant disease.

In a second aspect, the present invention relates to a composition comprising a γ-glutamyltransferase inhibitor for use in combination therapy with an NF-κB inhibitor for treating a malignant disease, preferably caused by TP53-mutated cells, in a human patient.

Accordingly, in embodiments of this second aspect, the invention also relates to a method of combination therapy of a malignant disease, preferably caused by TP53-mutated cells, comprising administering a γ-glutamyltransferase inhibitor in combination with an NF-κB inhibitor to a human patient, as well as to use of a γ-glutamyltransferase inhibitor in the manufacture of a medicament for combination therapy of a malignant disease, preferably caused by TP53-mutated cells, with an NF-κB inhibitor.

Preferably the disease in the embodiments of the present invention, and in both of the above first and second aspects, is a malignant disease caused by TP53-mutated cells. Generally, the disease is selected from the group consisting of: ovarian cancers; colorectal cancers; oesophageal cancers; head and neck cancers; laryngeal cancers; lung cancers; skin cancers; pancreatic cancers; stomach cancers; liver cancers; brain cancers; bladder cancers; breast cancers; uterus cancers; soft tissue cancers; leukaemias; lymphomas; prostate cancer; bone cancers; endocrine gland cancers; testicle cancers; kidney cancers; haematopoietic cancers; cervical cancers; cholangiocarcinoma; Li-Fraumeni syndrome; osteosarcoma;
rhabdomyocarcinoma; adrenocortical carcinoma; chronic lymphatic leukaemia (CLL); myelodysplastic syndromes (MDS); and acute myeloid leukaemia (AML).

In a third aspect, the present invention relates to an in vitro method of identifying an NF-κB inhibitor for its potential to act as a therapeutically effective NF-κB inhibitor in the treatment of a malignant disease, preferably caused by TP53-mutated cells, in vivo, said method comprising exposing an in vitro culture of malignant human cells comprising human serum to the NF-κB inhibitor and to a γ-glutamyltransferase inhibitor, wherein the NF-κB inhibitor is identified as having high potential to act as a therapeutically effective NF-κB inhibitor in the treatment of a malignant disease, preferably caused by TP53-mutated cells, in vivo, if a cytostatic and/or cytotoxic effect on the cells is observable.

Preferably, the method of the second aspect comprises the steps of:
(a) exposing an in vitroculture of malignant human cells to the NF-κB inhibitor and assessing the cytostatic and/or cytotoxic effect on the cells, wherein the culture does not contain human serum;
(b) exposing an in vitroculture of malignant human cells to the NF-κB inhibitor and assessing the cytostatic and/or cytotoxic effect on the cells, wherein the culture contains human serum;
(c) exposing an in vitro culture of malignant human cells corresponding to the culture of (b) to the NF-κB inhibitor in combination with a γ-glutamyltransferase inhibitor and assessing the cytostatic and/or cytotoxic effect on the cells; and
(d) comparing the cytostatic and/or cytotoxic effects assessed in (a), (b) and (c).

Preferably, when the NF-κB inhibitor's cytostatic and/or cytotoxic effect is decreased or unchanged in (b) compared to (a) but is restored or enhanced in (c), the NF-κB inhibitor is identified as having high potential to act as a therapeutically effective NF-κB inhibitor in the treatment of a malignant disease, preferably caused by TP53-mutated cells, in vivo. Preferably, in the method of the third aspect, the NF-κB inhibitor having high potential to act as a therapeutically effective NF-κB inhibitor in the treatment of a malignant disease, preferably caused by TP53-mutated cells, in vivo is selected for use in a composition of the first or second aspect.

In a fourth aspect, the present invention relates to a kit comprising a γ-glutamyltransferase inhibitor and an NF-κB inhibitor for use in the treatment of a malignant disease, preferably caused by TP53-mutated cells, in a human patient.

In a fifth aspect, the present invention relates to use of a γ-glutamyltransferase inhibitor for increasing the efficacy of an NF-κB inhibitor against TP53-mutated cells in vitro.

### FIGURES

Embodiments of the invention will now be described, by way of example only, with reference to the accompanying drawings in which:

FIG. 1 illustrates the clinical results obtained when leflunomide was administered to patients suffering from refractory CLL. A. In vitro detection of apoptosis of primary CLL cells upon administration of fludarabine or leflunomide at 80 and 120 μg/ml, cell culture with fetal calf serum (FCS). B. In vivo results of leflunomide administration in two patients. C. In vitro detection of apoptosis of primary CLL cells upon administration of leflunomide at 120 μg/ml, cell culture with either FCS or autologous human serum (HS).

FIG. 2 shows diminished NF-κB inhibitor activity of four NF-κB inhibitors on different tumour cell lines when cultured in medium containing HS compared to fetal calf serum (FCS).

FIG. 3 shows that the NF-κB inhibitor activity of four NF-κB inhibitors can be restored and/or enhanced in different tumour cell lines through the addition of a γ-GT inhibitor when cultured in medium containing HS.

FIG. 4 shows that the NF-κB inhibitor activity of NF-κB inhibitors can be restored and/or enhanced in the MEC1 cell line through the addition of glutathione catabolism inhibitors when cultured in medium containing HS.

FIG. 5 shows that the NF-κB inhibitor activity of NF-κB inhibitors can be restored and/or enhanced in the MOLM13 cell line through the addition of glutathione catabolism inhibitors when cultured in medium containing HS.

FIG. 6 shows that the addition of Cysteinyl-Glycin (CysGly) dipeptides to cell culture medium containing either FCS or HS increases the protective effect against NF-κB inhibition by BAY 11-7082 in different tumour cell lines. A. MEC1 cell line. B. MOLM13 cell line.

FIG. 7 shows that reduced ROS levels are detectable in MEC1 and MOLM13 cells when cultured in HS-containing cell culture medium compared to the levels detectable when the cells are cultured in FCS-containing cell culture medium.

FIG. 8 shows that increased ROS levels are detectable in MEC1 and MOLM13 cells when cultured in HS-containing cell culture medium and exposed to a γ-GT inhibitor.

FIG. 9 shows that the assessment of the total anti-oxidant capacity of various sera, albumin is as well as albumin-supplemented FCS.

FIG. 10 shows intracellular ROS as well as superoxide levels of in vitro cultured MOLM13 and MEC1 cells in relation to their extracellular environment; A: in relation to the cell culturing conditions: serum-free or 10% FCS-supplemented or 10% HS-supplemented culture medium; B: when cultured in 10% and 20% FCS-supplemented as well as 1.25%, 2.5%, 5% and 10% HS-supplemented culture medium; C: when cultured in 10% FCS-supplemented, 10% FCS+BSA-supplemented as well as 10% FCS+HSA-supplemented culture medium (BSA as well as HSA=approx. 4 mg/ml); and D: when cultured in 10% FCS-supplemented cell culture medium as well as medium supplemented with BSA or various different HSA preparations such as HSAph: pharmaceutical-grade HSA, HSAfaf: fatty-acid-free HSA; HSAlyo: commercial lyophilized HSA; HSArec: recombinant HSA.

FIG. 11 shows that the NF-κB inhibitor efficacy of four NF-κB inhibitors as indicated by their respective IC₅₀ concentrations is dose-dependently affected in both MOLM13 and MEC1 cells when cultured in medium supplemented with various concentrations and types of sera. A: when cultured in 10% and 20% FCS-supplemented as well as 1.25%, 2.5%, 5% and 10% HS-supplemented culture medium; B: when cultured in 10% FCS-supplemented, 10% FCS+BSAlow-supplemented as well as 10% FCS+BSAhigh-supplemented culture medium (BSAlow=approx. 4 mg/ml and BSAhigh=approx. 7 mg/ml); and C: when cultured in 10% FCS-supplemented, 10% FCS+HSAlow-supplemented as well as 10% FCS+HSAhigh-supplemented culture medium (HSAlow=approx. 4 mg/ml and HSAhigh=approx. 7 mg/ml)

FIG. 12 shows the differential effect of OU749 on the growth of both MOLM13 and MEC1 cells when cultured in 10% FCS-supplemented or 10% HS-supplemented culture medium.

FIG. 13 shows the enhanced effect of four NF-κB inhibitors on the growth of both MOLM13 and MEC1 cells when administered in combination with increasing concentrations of OU749, in particular 25, 50 and 100 μM of OU749. A: when cultured in 10% FCS-supplemented culture medium; B: when cultured in 10% HS-supplemented, culture medium C: when cultured in 10% FCS+BSAlow-supplemented culture medium (BSAlow=approx. 4 mg/ml); D: when cultured in 10% FCS+BSAhigh-supplemented culture medium (BSAhigh=approx. 7 mg/ml); E: when cultured in 10% FCS+HSAlow-supplemented culture medium (HSAlow=approx. 4 mg/ml); and F: when cultured in 10% FCS+HSAhigh-supplemented culture medium (HSAhigh=approx. 7 mg/ml).

FIG. 14 shows intracellular ROS as well as superoxide levels of in vitro cultured MOLM13 and MEC1 cells in relation to their extracellular environment; A: in relation to the cell culturing conditions: 10% FCS+DMSO, 10% FCS+100 μM of OU749, 10% HS+DMSO or 10% HS+200 μM of OU749-supplemented culture medium; B: when cultured in 20 mg/ml BSA+DMSO, 20 mg/ml BSA+200 μM of OU749, 20 mg/ml HSA+DMSO or 20 mg/ml HSA+200 μM of OU749.

FIG. 15 shows that the treatment of in vitro cultured MOLM13 and MEC1 cells with NF-κB inhibitors BAY 11-7082, EF24, leflunomide and sorafenib in combination with a γ-GT inhibitor increases the levels of intracellular ROS. A: when cultured in 10% FCS-supplemented; B: 10% HS-supplemented culture medium.

FIG. 16 shows that in both MOLM13 and MEC1 cells NF-κB activity depends on the cells extracellular environment as shown by the level of activation of NF-κB family member transcription factors p65, p50, c-Rel, p52, and RelB when cultured in serum-free or 10% FCS-supplemented or 10% HS-supplemented or 20 mg/ml HAS-supplemented culture medium.

FIG. 17 shows that the efficacy of NF-κB inhibitors BAY 11-7082 and EF24 in both MOLM13 and MEC1 depends on the cells extracellular environment. A: when cultured in 10% FCS-supplemented medium NF-κB inhibitors BAY 11-7082 and EF24 reduce NF-κB activity; B: when cultured in 10% HS-supplemented medium NF-κB inhibitors BAY 11-7082 and EF24 only reduce NF-κB activity when administered in combination with a γ-GT inhibitor.

FIG. 18 illustrates the protein interaction/signalling relationships relevant in the context of the present invention. A: protein interaction/signalling scheme when cells are cultured in a pro-oxidant environment where the cells display high susceptibility towards NF-κB inhibitors; B: protein interaction/signalling scheme when cells are cultured in an anti-oxidant environment where the cells display a low susceptibility towards NF-κB inhibitors; and C: protein interaction/signalling scheme when cells are cultured in an anti-oxidant environment but cells display a high susceptibility towards NF-κB inhibitors due to the action of the γ-GT inhibitor OU749. Abbreviations: Cys: cysteine; Cys-Gly: cysteinyl-glycine; γ-GT: γ-glutamyltransferase; Glu: glutamate; GSH: glutathione; GS-S-R, oxidized glutathione; ROS: reactive oxygen species; X_{c}: cystine transporter.

### DETAILED DESCRIPTION OF THE INVENTION

In order to provide a clear and consistent understanding of the specification and claims, as well as of the scope of particular terms used throughout, the following definitions are provided.

### Definitions

In the context of the present application, the term “NF-κB inhibitor” refers to molecules inhibiting the NF-κB signalling pathway, including direct and indirect NF-κB inhibitors, thereby leading to a down regulation of NF-κB induced gene expression. In the context of the present application, the term “NF-κB inhibitor” expressly includes NF-κB inhibitors with no, hitherto insufficient or unexplored clinical efficacy in the treatment of a malignant disease. However, as indicated above, if NF-κB inhibitors act as anti-tumour agents in vivo, their mechanism of inducing cytotoxicity in tumours differs from those of “chemotherapeutic agents” as defined below because NF-κB inhibitors specifically target the NF-κB signalling pathway. The class of NF-κB inhibitors is well known in the art and, as such, the term includes previously disclosed NF-κB inhibitors. A list of NF-κB inhibitors is provided at the NF-κB-dedicated Internet site www.nf-kb.org.

In the context of the present application, the term “γ-glutamyltransferase inhibitor” refers to molecules reducing or diminishing the activity of γ-glutamyltransferase (γ-GT). Such molecules may be occupying the γ-glutamyl site or the acceptor site of γ-GT and therefore include glutamine analogues, glutamyl analogues, sulphur derivatives of L-glutamic acid, γ-(monophenyl)phosphono glutamate analogues as well as N-[5-(4-methoxybenzyl)-1,3,4-thiadiazol-2-yl]benzenesulfonamide (OU749) and its γ-GT inhibitory derivatives. Particularly suitable γ-GT inhibitors in the context of the present invention are non-toxic, non-competitive γ-GT inhibitors such as OU749.

In the context of the present application, the term “malignant disease” refers to malignant diseases involving abnormal cell growth with the potential to invade or spread to other parts of the body, i.e. cancers. The malignant diseases referred to here are caused by “malignant cells”, which are deficient in mechanisms controlling cell growth, cell proliferation and/or cell differentiation (anaplasia) and are therefore responsible for the formation of tumours. Malignant cells are also capable of invading adjacent tissue (invasiveness) and may become capable of spreading to distant tissues (metastasis). Malignant cells routinely display a high degree of genome instability, and are therefore prone to having an increased rate of mutation, which in turn increases the chances of malignant cells to being or becoming resistant to chemotherapeutic agents.

With respect to the malignant diseases referred to in the present application, the phrase “caused by TP53 mutated cells” refers to malignant diseases having a mutation in the human p53/TP53 gene (NCBI Gene ID: 7157, updated on 16 Apr. 2017) leading to aberrant gene expression of TP53 protein regulated genes, wherein errant expression of those genes is predominantly responsible for the malignant disease phenotype.

In the context of the present application, the term “chemotherapeutic agents” refers to poisonous, cytotoxic agents taking advantage of malignant disease cells' rapid growth and consumption of large amounts of nutrients thereby preferentially killing malignant cells. However chemotherapeutic agents are equally poisonous for healthy cells leading to significant side-effects, which routinely include: severe nausea; temporary full or partial hair loss; bone marrow insufficiency causing infections; bleeding; diminished oxygen supply due to low haemoglobin; and/or kidney, liver or other organ damage. In addition, the term refers to agents already actively administered to cancer patients in a clinical setting but does not refer to pre-clinical cytotoxic agents.

In the context of the present application the term “chemotherapeutic agents” is distinguished from “anti-tumour agents”. Accordingly, anti-tumour agents include agents, such as antibodies and NF-κB inhibitors that do not require rapidly-growing and metabolically-active malignant cells but can also target quiescent cells. Therefore, NF-κB inhibitors are not to be considered chemotherapeutic agents in the context of the present application.

In the context of the present application the term “therapeutic efficacy” refers to clinically effects achieved through the administration of active agents, in particular anti-tumour agents in vivo leading to controlling or stopping the progression of a malignant disease, as well as to reversing or obliterating the malignant disease. However, the term also refers to effects achieved through the administration of active agents, in particular anti-tumour agents in in vitro models of malignant diseases, for example the effects cytostatic and/or cytotoxic effects of anti-tumour agents in cell culture systems of malignant diseases.

In addition to the above definitions, and unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Further, reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

As used herein, the term “exemplary” is used in the sense of providing examples, as opposed to indicating quality. That is, an “exemplary embodiment” is an embodiment provided as an example, as opposed to necessarily being an embodiment of exemplary quality.

### EMBODIMENTS OF THE INVENTION

As described above, the inventors observed that the promising anti-proliferative and pro-apoptotic effects of the NF-κB inhibitor leflunomide on clinically refractory CLL cells in vitro were lost when the NF-κB inhibitor was administered to CLL patients with TP53 mutations (Example 1). The administration of leflunomide to the CLL patients was such that leflunomide levels equivalent to those used in the in vitro cell culture assays were measurable in the patient's serum, i.e. at the site of the tumour in case of CLL. As such, the administration of amounts of leflunomide seen to have anti-proliferative effects and to induce apoptosis in vitro did not show therapeutic efficacy when administered to CLL patients, i.e. in vivo. While disappointing, this finding fits into the generally observed failure of NF-κB inhibitors with in vitro efficacy to achieve therapeutic efficacy in vivo.

The inventors have now not only found that the observed lack of in vivo efficacy of NF-κB inhibitors can be recreated in in vitro cell culture systems (Example 2) but also how the efficacy can be restored and/or enhanced in the same cell culture system—namely, by co-administration of a γ-GT inhibitor (Example 3) or an inhibitor of the glutathione catabolism (Examples 4) with the NF-κB inhibitor. In particular, it was surprisingly found that exchanging the fetal calf serum (FCS) component of the cell culture medium with human serum (HS) led to a significant decrease of the in vitro efficacy of NF-κB inhibitors (i.e. mirroring the regularly-observed, diminished in vivo efficacy of NF-κB inhibitors) but that NF-κB inhibitor efficacy can be restored and/or enhanced by co-administration of a γ-GT inhibitor or an inhibitor of the glutathione catabolism.

Without wanting to be bound by theory, reducing the amount of glutathione being produced in the cytosol appears to have an immediate influence on the efficacy of NF-κB inhibitors, i.e. on the ability of NF-κB inhibitors to exert cytostatic and cytotoxic effects on cancer cells, preferably on TP-53 mutated cells. Surprisingly, even NF-κB inhibitors, for which cytostatic and/or cytotoxic effects could not previously be demonstrated, can exert cytostatic and/or cytotoxic effects when administered in combination with a γ-GT inhibitor or an inhibitor of the glutathione catabolism in accordance with the present invention.

Namely, the inventors surprisingly found that the in vitro inhibition of proliferation and induction of apoptosis mediated by a number of NF-κB inhibitors, such as leflunomide, was strongly diminished in cell cultures supplemented with human serum (HS) but could be restored and/or enhanced by the inhibition of γ-GT and/or by the inhibition glutathione catabolism.

In this regard, it is noteworthy that the activity of NF-κB signalling pathway modulators involves and depends on cellular reactive oxygen species (ROS) homeostasis indicating a mutual cross-talk between ROS and NF-κB signalling (Bubici et al. 2006).

In light of the above, and given the central role of ROS in NF-κB signalling pathway activation, the inventors performed experiments further elucidating the mechanisms underlying the present invention also focusing on intracellular ROS homeostasis and factors contributing to the redox equilibrium in the extracellular environment (i.e. in the present in vitro system: serum-supplementation of cell culture medium). Furthermore, the enhancement of the efficacy of NF-kB signalling pathway inhibitors was investigated including assessing the inhibition of γ-GT dose-dependently and in different extracellular environments. Additionally, activation levels of different members of the NF-κB family of transcription factors were assessed in light of the respective extracellular environment and/or inhibition of γ-GT activity.

The thiol/disulfide homeostasis plays a pivotal role in maintaining redox homeostasis. In cells, thiols are highly reduced and present at millimolar concentrations. In contrast, in the extracellular environment, and particularly in the serum/plasma compartment, reduced thiol concentrations are much lower. Human serum albumin (HSA) constitutes the most abundant reduced thiol (˜0.6 mM, up to 75% reduced). HSA is the major and predominant anti-oxidant in serum/plasma and is a central contributor to redox processes (Turell et al., 2013). In the context of the present invention, it is relevant that the albumin concentrations in commercial fetal calf serum (10-15 g/L, depending on source) are significantly lower as compared to human serum (30-50 g/L, reference range for a healthy adult). Accordingly, the inventors investigated the total anti-oxidant capacity of different serum and albumin preparations and albumin-supplemented FCS. Compared to FCS, the total anti-oxidant capacity was significantly higher in HS or in albumin-supplemented FCS. It was also higher for HSA as compared to bovine serum albumin (BSA).

Since the extracellular anti-oxidant capacity can actively affect cellular processes, the inventors also analysed basal intracellular ROS levels in the context of different extracellular environments in vitro. Addition of serum or defined albumin concentrations to serum-free cell culture medium suppresses the generation of intracellular ROS. Compared to FCS-supplemented media, the induction of ROS is significantly and dose-dependently suppressed in the presence of HS or albumin. Different HSA preparations (pharmaceutical-grade HSA, fatty-acid-free HSA, commercial lyophilized HSA and recombinant HSA) exert similar effects on intracellular ROS levels and are more potent than BSA in this regard.

With respect to the anti-proliferative activity of inhibitors of the NF-kB signalling pathway (such as BAY 11-7082, EF24, leflunomide and sorafenib) in in vitro cell culture systems supplemented with increasing HS concentrations, the sensitivity of cells towards NF-kB inhibitors is dose-dependently reduced as reflected by the significantly increased IC₅₀ concentrations of the inhibitors. Further, compared to FCS, the addition of HSA or BSA to FCS-supplemented cell culture systems also reduces the sensitivity of cells towards NF-kB inhibitors.

Inhibition of γ-GT activity by OU749 exerts no effects on cell proliferation in HS-supplemented cells systems in doses up to 200 μM. However, in contrast, in FCS-supplemented culture systems, the anti-proliferative effects of single-agent OU749 are discernible above doses of 50 μM. To further investigate the enhancement of the efficacy of NF-kB signalling pathway inhibitors by inhibition of γ-GT activity in more detail, the inventors assessed the anti-proliferative effects of NF-kB inhibitors (such as BAY 11-7082, EF24, leflunomide and sorafenib) in different extracellular environments and in the context of dose-dependent OU749 treatment. Addition of OU749 dose-dependently enhances the sensitivity of cells towards NF-kB inhibitors both in HS- but also in FCS-supplemented cell systems. In the context of HS and FCS, this effect is cell line dependent and was significant for OU749 doses 100 μM and 50 μM, respectively. Importantly, OU749 also enhances the anti-proliferative activity of NF-kB inhibitors in the context of FCS supplemented with albumins (BSA or HSA).

As regards intracellular ROS levels, the inhibition of γ-GT by OU749 increases intracellular ROS levels in the presence of serum (both FCS and HS) and albumin (both BSA and HSA). In HS-supplemented culture systems, treatment with OU749 restores ROS levels to the extent observed in FCS-supplemented culture systems. Treatment of cells with inhibitors of the NF-kB signalling pathway (BAY 11-7082, EF24, leflunomide) induces ROS in FCS-supplemented culture systems. In contrast, in the presence of HS, these inhibitors have only modest effect on intracellular ROS levels. However, after treatment with non-toxic doses of OU749, an increase in ROS levels is observed upon treatment with NF-kB inhibitors both in FCS- and, importantly, in HS-supplemented culture systems.

On the cellular level, presence of HS results in lower NF-κB pathway activation as compared to FCS-supplemented or serum-free culture conditions, which is reflected by significantly lower nuclear levels of different members of the NF-κB family of transcription factors (p65, p50, c-Rel, p52 and RelB). In the presence of FCS, inhibitors of the NF-kB signalling pathway (BAY 11-7082 and EF24) significantly reduces the nuclear levels of different NF-κB transcription factors indicating inhibition of NF-kB transactivation. In contrast, in the presence of HS no reduction of nuclear levels of NF-κB transcription factors is observed upon treatment of cells with BAY 11-7082 and EF24. Treatment with non-toxic doses of OU749 results in increased nuclear levels of different members of the NF-κB family of transcription factors indicating NF-κB pathway activation. Importantly, after treatment with non-toxic doses of OU749, a decrease in nuclear levels of members of NF-κB family of transcription factors is observed upon treatment with NF-kB inhibitors in HS-supplemented culture systems indicating inhibition of NF-kB transactivation in HS in the context of γ-GT inhibition.

In conclusion, based on the experiments performed, and in light of the mutual cross-talk between cellular ROS homeostasis and NF-κB signalling, the inventors considered that basal intracellular ROS levels are linked to the anti-oxidant capacity of the extracellular environment. Without wanting to be bound by theory, free (reduced) thiol content of the extracellular environment (i.e. serum/albumin concentration in the cell culture system) may be the major determinant of this anti-oxidant capacity. Low anti-oxidant capacity of the extracellular environment (i.e. lower concentrations of serum/albumin in the cell culture system) is associated with increased intracellular ROS levels and higher basal activity of the NF-kB signalling pathway. These higher basal activity of the NF-kB signalling pathway in turn renders neoplastic cells more susceptible towards inhibitors of the NF-kB signalling pathway and results in cell growth inhibition and apoptosis upon treatment with NF-kB inhibitors.

In contrast, higher anti-oxidant capacity of the extracellular environment (i.e. higher free thiol content due to higher concentrations of serum/albumin in the cell culture system) results in the suppression of intracellular ROS levels and lower basal activity of the NF-kB signalling pathway. Consequently, lower basal NF-kB signalling pathway activation results in lower sensitivity of neoplastic cells towards inhibitors of the NF-kB signalling pathway and leads to lower efficacy of NF-kB inhibitors in an anti-oxidant extracellular environment.

Inhibition of γ-GT activity by OU749 increases basal intracellular ROS levels regardless of the anti-oxidant environment and consequently results in higher basal activity of the NF-kB signalling pathway. Higher basal activity of the NF-kB signalling pathway upon treatment of neoplastic cells with OU749 thereby restores/enhances the efficacy of NF-kB signalling pathway inhibitors in an anti-oxidant environment. This is of particular importance in the context of a HS-supplemented cell culture system, which more adequately reflects the in vivo situation in patients, thereby bearing high relevance for achievement therapeutic efficacy of NF-kB inhibitors in vivo.

Accordingly, in the first aspect, the present invention relates to a composition comprising an NF-κB inhibitor and a γ-GT inhibitor.

In one or more preferred embodiments of the invention, the γ-GT inhibitor is an inhibitor occupying the γ-GT γ-glutamyl site, such as a glutamine analogue, a glutamyl analogue, a sulphur derivative of L-glutamic acid, a γ-(monophenyl)phosphono glutamate analogue. In alternative embodiments, the γ-GT inhibitor is an inhibitor occupying the γ-GT acceptor site, such as N-[5-(4-methoxybenzyl)-1,3,4-thiadiazol-2-yl]benzenesulfonamide (OU749) and its γ-GT inhibitory derivatives. Most preferably, the γ-GT inhibitor is a non-toxic, non-competitive γ-GT inhibitor, namely OU749.

In one or more preferred embodiments of the invention, the NF-κB inhibitor is selected from the group consisting of: BAY 11-7082; EF24; leflunomide; sorafenib; bortezomib; thalidomide; lenalidomide; arsenic trioxide; and ibrutinib. Preferably, the NF-κB inhibitor is leflunomide or sorafenib. Most preferably, the NF-κB inhibitor is leflunomide.

In one or more preferred embodiments, the NF-κB inhibitor is not an NF-κB inhibitor for which in vivo efficacy in the treatment of a malignant disease caused by TP53-mutated cells was previously demonstrated. In one or more alternative embodiments, the NF-κB inhibitor is not bortezomib, thalidomide, lenalidomide, arsenic trioxide, sorafenib or ibrutinib.

In alternative embodiments where an inhibitor of the glutathione catabolism is administered in combination with a γ-GT inhibitor, the inhibitor of the glutathione catabolism is selected from the group of: buthionine sulfoximine (BSO); auranofin; and sulfasalazine. Sulfasalazine blocks cystine uptake through System Xc- (an amino acid antiporter that typically mediates the exchange of extracellular l-cystine and intracellular l-glutamate) thereby leading to glutathione depletion.

As explained above, the compositions of the invention are particularly suitable for use in medical treatment. Typically, the compositions of the invention are particularly suitable for use in the treatment of a malignant disease, preferably caused by TP53-mutated cells.

Accordingly, in embodiments of this aspect, the invention also relates to methods of medical treatment, such as the treatment of a malignant disease, comprising the administration of an NF-κB inhibitor and a γ-GT inhibitor as well as to use of an NF-κB inhibitor and a γ-GT inhibitor in the manufacture of a medicament for treating a malignant disease.

Preferably, the malignant disease in the embodiments of the present invention is selected from the group consisting of: ovarian cancers; colorectal cancers; oesophageal cancers; head and neck cancers; laryngeal cancers; lung cancers; skin cancers; pancreatic cancers; stomach cancers; liver cancers; brain cancers; bladder cancers; breast cancers; uterus cancers; soft tissue cancers; leukaemias; lymphomas; prostate cancer; bone cancers; endocrine gland cancers; testicle cancers; kidney cancers; haematopoietic cancers; cervical cancers; cholangiocarcinoma; Li-Fraumeni syndrome; osteosarcoma; rhabdomyocarcinoma; adrenocortical carcinoma; chronic lymphatic leukaemia (CLL); myelodysplastic syndromes (MDS); and acute myeloid leukaemia (AML).

In particular preferred embodiments of the present invention, the malignant disease is selected from TP53-mutated leukaemias, lymphomas, ovarian cancers, breast cancers, lung cancers and pancreatic cancers.

In particular embodiments of the present invention the TP53-mutated cells are refractory to a chemotherapeutic agent. For example, the disease is a chronic disease, such as chronic lymphatic leukaemia (CLL). The CLL may be caused by TP53-mutated cell and may be refractory to chemotherapeutic treatment, such as to treatment with fludarabine and cyclophosphamide or bendamustine. Alternatively, the disease is an acute disease, such as acute myeloid leukaemia (AML). The AML may be caused by TP53-mutated cell and may be refractory to chemotherapeutic treatment, such as to treatment with cytarabine and daunorubicin or mitoxantrone.

In a second aspect, the present invention relates to a composition comprising a γ-GT inhibitor for use in combination therapy with an NF-κB inhibitor for treating a malignant disease, preferably caused by TP53-mutated cells, in a human patient.

Accordingly, in embodiments of this second aspect, the invention relates to a method of combination therapy of a malignant disease, preferably caused by TP53-mutated cells, comprising administering a γ-GT inhibitor in combination with an NF-κB inhibitor to a human patient, as well as to use of a γ-GT inhibitor in the manufacture of a medicament for combination therapy with an NF-κB inhibitor.

Preferably, the NF-κB inhibitor is therapeutically effective in the combination therapy and/or the therapeutic efficacy of the NF-κB inhibitor is increased in the combination therapy. For example, the therapeutic efficacy of leflunomide may be increased by: more than 1.25-fold, such as, e.g., by between 1.25 and 10-fold; or by more than 1.5-fold, such as, e.g., by between 1.5- and 10-fold; or by more than 2-fold, such as, e.g., by between 2- and 10-fold; or by more than 2.5-fold, such as, e.g., by between 2.5- and 10-fold; or by more than 3-fold, such as, e.g., by between 3- and 10-fold; or by more than 3.5-fold, such as, e.g., by between 3.5- and 10-fold; or by more than 4-fold such as, e.g., by between 4- and 10-fold; or by about 3.5-fold when administered in combination therapy with OU749.

In one or more preferred embodiments, the NF-κB inhibitor is an NF-κB inhibitor previously-shown to be ineffective in in vivo treatment of a malignant disease such as a disease caused by TP53-mutated cells in vivo, but which achieves therapeutic efficacy in the combination therapy. For example, the NF-κB inhibitor is selected from the group consisting of: BAY 11-7082; EF24; leflunomide; sorafenib; bortezomib; thalidomide; lenalidomide; arsenic trioxide; and ibrutinib. Preferably, the NF-κB inhibitor is leflunomide or sorafenib. Most preferably, the NF-κB inhibitor is leflunomide. Preferably, the NF-κB inhibitor is cytostatic or cytotoxic to the TP53-mutated cells when used in the combination therapy.

In one or more preferred embodiments, the NF-κB inhibitor is not an NF-κB inhibitor for which in vivo efficacy in the treatment of a malignant disease caused by TP53-mutated cells was previously demonstrated. In one or more preferred embodiments, the inhibitor is not bortezomib, thalidomide, lenalidomide, arsenic trioxide, sorafenib, or ibrutinib.

In one or more preferred embodiments of the invention, the γ-GT inhibitor is an inhibitor occupying the γ-GT γ-glutamyl site, such as a glutamine analogue, a glutamyl analogue, a sulphur derivative of L-glutamic acid, a γ-(monophenyl)phosphono glutamate analogue. In alternative embodiments, the γ-GT inhibitor is an inhibitor occupying the γ-GT acceptor site, such as N-[5-(4-methoxybenzyl)-1,3,4-thiadiazol-2-yl]benzenesulfonamide (OU749) and its γ-GT inhibitory derivatives. Most preferably, the γ-GT inhibitor is a non-toxic, non-competitive γ-GT inhibitor, namely OU749.

In alternative embodiments where an inhibitor of the glutathione catabolism is administered in combination with a γ-GT inhibitor, the inhibitor of the glutathione catabolism is selected from the group of: buthionine sulfoximine (BSO); auranofin; and sulfasalazine.

Preferably, the disease is selected from the group consisting of: ovarian cancers; colorectal cancers; oesophageal cancers; head and neck cancers; laryngeal cancers; lung cancers; skin cancers; pancreatic cancers; stomach cancers; liver cancers; brain cancers; bladder cancers; breast cancers; uterus cancers; soft tissue cancers; leukaemias; lymphomas; prostate cancer; bone cancers; endocrine gland cancers; testicle cancers; kidney cancers; haematopoietic cancers; cervical cancers; cholangiocarcinoma; Li-Fraumeni syndrome; osteosarcoma; rhabdomyocarcinoma; adrenocortical carcinoma; chronic lymphatic leukaemia; (CLL); myelodysplastic syndromes (MDS) and acute myeloid leukaemia (AML).

The compositions of the present invention can formulated for administration to a patient via a wide range of administration routes. By way of example but not limitation, suitable routes of administration include enteric, parenteral, topical, oral, rectal, nasal, and/or vaginal routes. Parenteral routes include subcutaneous, intramuscular, intravenous, intraperitoneal, intradermal, and subilingual administration. Also, compositions may be implanted and/or injected into a patient using a drug delivery system. The compositions may be administered locally and/or systemically. The term “systemic administration” refers to any mode or route of administration that result in effective amounts of NF-κB inhibitor appearing in the blood and/or at a site remote from the initial administration.

The compositions of the invention when formulated for oral administration can be, but are not limited to, (a) solid dosage forms such as capsules, tablets, pills, powders, troches, and granules, and (b) liquid dosage forms such as pharmaceutically acceptable emulsions, solutions, suspensions, syrups, and elixirs. When formulated as injectable solutions the compositions include, but are not limited to, intravenous, subcutaneous, and intramuscular injectable solutions.

The dosage levels of the γ-GT inhibitor and/or the NF-κB inhibitor in the compositions of the invention may be varied so as to obtain an amount of the NF-κB inhibitor effective to achieve in vivo efficacy in accordance with the desired method of administration. The selected dosage level therefore depends upon the nature of the particular NF-κB inhibitor to be administered, the route of administration, the desired duration of treatment, individual needs, and other factors. If desired, the compositions may be such that the daily requirement for NF-κB inhibitor is in one dose, or divided among multiple doses for administration, e.g., two to four times per day.

The dosage of the γ-GT inhibitor and the NF-κB inhibitor may be varied depending on age, weight, symptoms, therapeutic effects, administration route, treatment time, and other factors. With regard to dosage and duration of treatment, the skilled person is able to determine suitable dosages depending on the relevant factors to be considered and monitors the patient such as to determine whether administration of the compositions of the present invention is to be started, continued, discontinued, or resumed at any given time.

For example, dosages of the compounds can be suitably determined depending on the symptoms of the individual patient, his/her weight, age, and sex and other relevant factors. The amount of the compound to be incorporated into the compositions of the present invention varies with the solubility of the γ-GT and NF-κB inhibitors, the route of administration, the administration schedule, and the like. An effective amount for a particular patient may vary depending on the malignant disease being treated, the overall health of the patient, and the method, route, and dose of administration.

A clinician can use parameters known in the art to determine the appropriate dose. Generally, the dose begins with an amount somewhat less than the optimum dose, and it is increased by small increments thereafter until the desired or optimum effect is achieved. Suitable dosages can be determined by further taking into account relevant disclosures known in the art.

Accordingly, the compositions and kits of the present invention are formulated such that administration results in blood serum concentrations of between 1 and 270 μg/ml of the NF-KB inhibitor and between 1 and 400 μg/ml of the γ-GT inhibitor. In particular, administration results in blood serum concentrations of between 100 and 250 μg/ml of the NF-κB inhibitor and between 10 and 150 μg/ml of the γ-GT inhibitor.

In some embodiments, the compositions and kits of the present invention are formulated such that administration results in blood serum concentrations of between 4 and 1000 μM of the NF-κB inhibitor and between 3 and 1000 μM of the γ-GT inhibitor. In particular, administration results in blood serum concentrations of between 370 and 650 μM of the NF-κB inhibitor and between 30 and 400 μM of the γ-GT inhibitor. In some embodiments, the compositions of the present invention can be formulated for intravenous administration such that doses of more than 50 mg/m²/day, or more than 60 mg/m²/day, or more than 70 mg/m²/day, or more than 80 mg/m²/day, or more than 90 mg/m²/day, or more than 100 mg/m²/day, or more than 110 mg/m²/day, or more than 120 mg/m²/day, or more than 130 mg/m²/day, or more than 140 mg/m²/day, or more than 150 mg/m²/day, of the γ-GT inhibitor can be administered. In other preferred embodiments the compositions are formulated such that more than 30 mg/m²/day of the γ-GT inhibitor can be administered in a dosage regime of intravenous administration over 30 minutes daily for 5 consecutive days. The compositions of the present invention are formulated to result in peak plasma concentrations of the γ-GT inhibitor of more than 2 μg/ml, preferably more than 2.5 μg/ml, preferably more than 3 μg/ml, preferably more than 3.5 μg/ml, preferably more than 4 μg/ml, preferably more than 4.5 μg/ml preferably more than 5 μg/ml.

In embodiments of the third aspect, the present invention relates to an in vitro method of identifying an NF-κB inhibitor for its potential to act as a therapeutically effective NF-κB inhibitor in the treatment of a malignant disease, preferably caused by TP53-mutated cells, in vivo. As indicated above, and in particular when considering the vast number of available NF-κB inhibitors for which in vitro efficacy has been shown, and the significant costs associated with bringing a promising pre-clinical drug candidate to the clinic, the here-described in vitro method of identifying an NF-κB inhibitor for its potential to act as a therapeutically effective NF-κB inhibitor in the treatment of a malignant disease in vivo addresses a long-felt need in the art and constitutes significant progress towards providing a whole suite of new therapeutic interventions in the treatment of cancer.

In particular, the in vitro method comprises exposing an in vitro culture of malignant human cells comprising human serum to the NF-κB inhibitor and to a γ-glutamyltransferase inhibitor, wherein the NF-κB inhibitor is identified as having high potential to act as a therapeutically effective NF-κB inhibitor in the treatment of a malignant disease, preferably caused by TP53-mutated cells, in vivo, if a cytostatic and/or cytotoxic effect on the cells is observable.

In one or more preferred embodiments, the in vitro method comprises of the steps of:
(a) exposing an in vitroculture of malignant human cells to the NF-κB inhibitor and assessing the cytostatic and/or cytotoxic effect on the cells, wherein the culture does not contain human serum;
(b) exposing an in vitroculture of malignant human cells to the NF-κB inhibitor and assessing the cytostatic and/or cytotoxic effect on the cells, wherein the culture contains human serum;
(c) exposing an in vitro culture of malignant human cells corresponding to the culture of (b) to the NF-κB inhibitor in combination with a γ-GT inhibitor and assessing the cytostatic and/or cytotoxic effect on the cells; and
(d) comparing the cytostatic and/or cytotoxic effects assessed in (a), (b) and (c).

Preferably, when the NF-κB inhibitor's cytostatic and/or cytotoxic effect is decreased or unchanged in (b) compared to (a) but is restored or enhanced in (c), the NF-κB inhibitor is identified as having high potential to act as a therapeutically effective NF-κB inhibitor in the treatment of a malignant disease, preferably caused by TP53-mutated cells, in vivo. In particular, the NF-κB inhibitor is identified as having high potential to act as a therapeutically effective NF-κB inhibitor in the treatment of a malignant disease in a human patient when administered in combination with the γ-GT inhibitor.

Alternatively, when the NF-κB inhibitor's cytostatic and/or cytotoxic effect is not decreased in (b) compared to (a) and is maintained in (c), the NF-κB inhibitor has a high potential to act as a therapeutically effective NF-κB inhibitor in the treatment of a malignant disease in a human patient, preferably when administered as a monotherapy.

In particular embodiments of the above method the in vitro culture of human cells of steps (b) and (c) above contains human serum from a particular patient suffering from the malignant disease to be treated. As such, depending on the comparison of step (d), the method of the present invention provides an invaluable tool for identifying NF-κB inhibitors with a high potential for in vivo efficacy in the treatment of the particular, individual cancer patient. As will be appreciated, the present method allows for a further level of personalised tailoring of the therapeutic regime to be employed when treating a patient suffering from a malignant disease.

Accordingly, in embodiments of the methods of the third aspect the NF-κB inhibitor having high potential to act as a therapeutically effective NF-κB inhibitor in the treatment of a malignant disease, preferably caused by TP53-mutated cells, in vivo is selected for use in a composition according to any one of the embodiments of the first and second aspects described above.

Preferably, the malignant disease is caused by TP53-mutated cells and, optionally, the γ-GT inhibitor is OU749 and/or the NF-κB inhibitor is selected from the group consisting of: BAY 11-7082; EF24; leflunomide; sorafenib; bortezomib; thalidomide; lenalidomide; arsenic trioxide; and ibrutinib. Preferably, the NF-κB inhibitor is leflunomide or sorafenib. Most preferably, the NF-κB inhibitor is leflunomide.

In one or more preferred embodiments, the NF-κB inhibitor identified in the methods of the third aspect is not an NF-κB inhibitor for which in vivo efficacy in the treatment of a malignant disease caused by TP53-mutated cells was previously demonstrated. In one or more preferred embodiments, the inhibitor is not bortezomib, thalidomide, lenalidomide, arsenic trioxide, sorafenib or ibrutinib.

In preferred embodiments, the cytostatic effect is assessed in steps (a) to (c) by assessing cell proliferation and/or by assessing induction of apoptosis.

Accordingly, in embodiments of the fourth aspect, the present invention also relates to a kit comprising a γ-GT inhibitor and an NF-κB inhibitor for use in the treatment of a malignant disease, preferably caused by TP53-mutated cells, in a human patient. In some preferred embodiments, the kits of the present invention comprise compositions of the γ-GT inhibitor for intravenous administrations and compositions of the NF-κB inhibitor for oral administration. In some embodiments, the kits are designed and/or constructed such as to ensure that γ-GT inhibitor and the NF-κB inhibitor are administered simultaneously or sequentially.

Preferably the γ-GT inhibitor is OU749 and/or the NF-κB inhibitor is selected from the group consisting of: BAY 11-7082; EF24; leflunomide; sorafenib; bortezomib; thalidomide; lenalidomide; arsenic trioxide; and ibrutinib and, preferably, the disease is selected from the group consisting of: ovarian cancers; colorectal cancers; oesophageal cancers; head and neck cancers; laryngeal cancers; lung cancers; skin cancers; pancreatic cancers; stomach cancers; liver cancers; brain cancers; bladder cancers; breast cancers; uterus cancers; soft tissue cancers; leukaemias; lymphomas; prostate cancer; bone cancers; endocrine gland cancers; testicle cancers; kidney cancers; haematopoietic cancers; cervical cancers; cholangiocarcinoma; Li-Fraumeni syndrome; osteosarcoma; rhabdomyocarcinoma; adrenocortical carcinoma; chronic lymphatic leukaemia (CLL); myelodysplastic syndromes (MDS) and acute myeloid leukaemia (AML).

Further, the present invention relates to use of a γ-GT inhibitor for increasing the efficacy of an NF-κB inhibitor against TP53-mutated cells in vitro. Preferably the γ-GT inhibitor is OU749.

### EXAMPLES

### Cell Culture

MEC1 Cells

The MEC1 cell line is a human chronic B cell leukaemia cell line established in 1993 from the peripheral blood of a 61-year-old Caucasian man with chronic B cell leukaemia (B-CLL in prolymphocytoid transformation to B-PLL).

According to flow cytometry data MEC1 cells are: CD3−, CD10−, CD13−, CD19+, CD20+, CD34−, CD37+, CD38+, cyCD79a+, CD80+, CD138+, HLA-DR+. The published karyotype is described as a human near-diploid karyotype with 10% polyploidy—46(44−47)<2n>XY, −2, +7, −12, +1−2mar, t(1;6)(q22-23;p21), add(7)(q11), der(10)(10pter->q22::?::2q11->qter), del(17)(p11). MEC1 cells are TP53-mutated (deleterious TP53 mutation resulting in truncated version of p53).

Cells were grown in RPM1-1640 medium supplemented with 10% heat inactivated FCS, 2 mM L-glutamine and 1% penicillin/streptomycin at standard conditions (37° C., fully humidified atmosphere of 95% air and 5% CO2). The optimal cell density in culture is around 0.5-2.0×10⁶ cells/ml. The cells are split twice weekly at a ratio of 1:5 to 1:10.

For assessment of cytostatic and/or cytotoxic effects MEC1 cells were washed in serum-free RPMI-1640 medium supplemented with 2 mM L-glutamine and 1% penicillin/streptomycin and diluted to 3.70×10⁵/ml (logarithmic cell growth).

Cells were incubated with BSO, auranofin, or combinations of both, and/or with OU749 for 10 minutes prior to addition of serum, albumin (FCS or HS, both 10% or different HSA preparations as indicated) or albumin-supplemented FCS (supplementation with HSA or BSA: final albumin concentration 4-5 mg/ml for FCS+BSA/HSAlow, or ˜7 mg/ml for FCS+BSA/HSAhigh) to washed cells resulting in a density of 3.33×10⁵/ml. For metabolic activity assays, here MTT-assays, cell density was 3×10⁴ cells per well (96 well plate). For ROS/superoxide detection assays cell density was 1×10⁵ per tube (volume 1 ml). For NF-κB family member activation assays nuclear cell extracts were used (9×10⁶ cells/6 ml volume in 6 well plates).

MOLM13 Cells

The MOLM13 cell line is a human acute myeloid leukaemia (AML) cell line established from the peripheral blood of a 20-year-old man with AML FAB M5a at relapse in 1995 after initial myelodysplastic syndromes (MDS, refractory anaemia with excess of blasts, RAEB). MOLM13 cells carry an internal tandem duplication of the fms like tyrosine kinase 3 (FLT3).

According to flow cytometry data MOLM13 cells are: CD3−, CD4+, CD13(+), CD14−, CD15+, CD19−, CD33+, CD34−, cyCD68+, HLA-DR−. The published karyotype is described as a human hyperdiploid karyotype with 4% polyploidy—51(48-52)<2n>XY, +8, +8, +8, +13, del(8)(p1?p2?), ins(11;9)(q23;p22p23). MOLM13 cells have a wild-type genotype for TP53.

Cells were grown in RPM1-1640 medium supplemented with 10% heat inactivated FCS, 2 mM L-glutamine and 1% penicillin/streptomycin at standard conditions (37° C., fully humidified atmosphere of 95% air and 5% CO2). The optimal cell density in culture is around 0.4-2.0×10⁶ cells/ml. The cells are split twice weekly at a ratio of 1:5 to 1:10.

For assessment of cytostatic and/or cytotoxic effects MOLM13 cells were washed in serum-free RPMI-1640 medium supplemented with 2 mM L-glutamine and 1% penicillin/streptomycin and diluted to 3.70×10⁵/ml (logarithmic cell growth).

Cells were incubated with BSO, auranofin, or combinations of both, and/or with OU749 for 10 minutes prior to addition of serum, albumin (FCS or HS, both 10% or different HSA preparations as indicated) or albumin-supplemented FCS (supplementation with HSA or BSA: final albumin concentration 4-5 mg/ml for FCS+BSA/HSAlow, or ˜7 mg/ml for FCS+BSA/HSAhigh) to washed cells resulting in a density of 3.33×10⁵/ml. For metabolic activity assays, here MTT-assays, cell density was 3×10⁴ cells per well (96 well plate). For ROS/superoxide detection assays cell density was 1×10⁵ per tube (volume 1 ml). For NF-KB family member activation assays nuclear cell extracts were used (9×10⁶ cells/6 ml volume in 6 well plates).

HL60 Cells

The HL60 cell line is a human AML cell line established from the peripheral blood of a 35-year-old woman with AML (AML FAB M2) in 1976.

According to flow cytometry data HL60 cells are: CD3−, CD4+, CD13+, CD14−, CD15+, CD19−, CD33+, CD34−, HLA-DR−. The published karyotype is described as a human flat-moded hypotetraploid karyotype with hypodiploid sideline and 1.5% polyploidy—82-88<4n>XX, −X, −X, −8, −8, −16, −17, −17, +18, +22, +2mar, ins(1;8)(p?31;q24hsr)×2, der(5)t(5;17)(q11;q11)×2, add(6)(q27)×2, der(9)del(9)(p13)t(9;14)(q?22;q?22)×2, der(14)t(9;14)(q?22;q?22)×2, der(16)t(16;17)(q22;q22)×1-2, add(18)(q21). HL60 cells are TP53-mutated (p53 null mutations lacking the entire p53 gene due to gene deletion or rearrangement).

Cells were grown in RPM1-1640 medium supplemented with 10% heat inactivated FCS, 2 mM L-glutamine and 1% penicillin/streptomycin at standard conditions (37° C., fully humidified atmosphere of 95% air and 5% CO2). The optimal cell density in culture is about 1×10⁶ cells/ml. The cells are split twice weekly at a ratio of 1:5 to 1:10.

For assessment of cytostatic and/or cytotoxic effects HL60 cells were washed in serum-free RPMI-1640 medium supplemented with 2 mM L-glutamine and 1% penicillin/streptomycin and diluted to 1.25×10⁵/ml (logarithmic cell growth). Cells were incubated with OU749 for 10 minutes prior to addition of serum (FCS or HS, both 10%) to washed cells resulting in a density of 1.11×10⁵/ml. For MTT-assays cell density was 1×10⁴ cells per well (96 well plate).

Assays for the Assessment of the Cytostatic and/or Cytotoxic Effects in Cell Lines

Cell Growth

The MTT or XTT assay is the most reliable for assessment of cell growth. In principal all assays are based on staining of viable cells after treatment. Cytotoxic effects in cell lines may also be assessed by [³H] thymidine incorporation (see below). Cell proliferation was assessed by the MTT assay. After washing in serum-free medium, pre-incubation with inhibitors of the glutathione catabolism, and addition of defined serum or albumin or serum+albumin concentrations, 90-μl aliquots of the cell suspension (density see above) were dispensed into 96-well flat-bottomed microtitre plates containing 10 μl/well of serial drug dilutions. The plates were incubated for 72 h under standard conditions. After addition of 15 μl of a 5 mg/ml MTT solution (MTT; Sigma, Germany) to each well and incubation for another 4 h, 100 μl/well of a 10% sodium dodecyl sulphate solution in 0.01 M hydrochloric acid were added and the plates were incubated overnight under the same conditions (stain development). Subsequently, the absorbance of each well at 540 nm (reference wavelength 690 nm) was recorded with an absorbance plate reader (Sunrise, Tecan, Switzerland). Blank control values (no drug, no cells) were subtracted from the sample values and the means of replicate wells for each drug dilution and the control (cells grown in absence of the drug) were used to calculate the extent of relative cell proliferation (as percentage of the control) and growth inhibition. Since inhibitors were initially dissolved in DMSO, serial drug dilutions and controls were prepared to finally achieve 0.2% DMSO in all wells (total volume 100 μl); no anti-proliferative effects could be observed at this DMSO concentration in the investigated cell lines.

Cell Proliferation Essay for Primary CLL Cells

CLL cells are seeded at a concentration of 10⁵ per well in 96-well plates in triplicates for 96 hours and stimulated with CD40L/IL 4. Cells were pulsed with 0.5 μCi (0.0185 MBq) per well [³H] thymidine (TdR; Hartmann Analytik) for the last 16 hours of culture and harvested on a semiautomatic cell harvester (Tomtec). [³H]TdR incorporation was quantified in a TopCount Scintillation Counter (Perkin-Elmer).

Detection of Apoptosis by Flow Cytometry

Apoptotic cell death is detected by flow cytometry with Annexin V-propidium iodide (PI) or 7-amino-actinomycin (7-AAD) staining. Cells are harvested and resuspended in Annexin V-binding buffer (10 mmol/L HEPES/NaOH, pH 7.4, 140 mmol/L NaCl and 2.5 mmol/L CaCl2)) containing 10% Annexin V-fluorescein isothiocyanate and 10% PI staining solution, or 10% Annexin V-PE and 10% 7-AAD staining solution (BD Biosciences). After an incubation time of 15 minutes at 4° C., stained cells are analysed by flow cytometry gating on lymphocytes. Double-negative cells are counted as viable cells. The results are confirmed on the basis of changes in forward light scattering properties of dead cells that have decreased cell size.

Western Blot Analysis

Cells are exposed to the NF-κB inhibitor and/or the γ-GT inhibitor as indicated, harvested, washed, resuspended, and lysed at a density of approximately 3×10⁷ cells per millilitre in Western Blot Sample Buffer (50 mmol/L Tris, pH 7.5, 1% BriJ 96V, 10 mmol/L NaF, 1 mmol/L Na-orthovanadate, 1 μg/ml leupeptin, 1.5 μg/ml pepstatin, 100 mmol/L phenylmethanesulfonylfluoride) and snap frozen. Protein concentrations of the lysates are determined by a modified Bradford method (Bio-Rad Laboratories). Cell lysates are analysed by SDS-PAGE and Western blots with antibodies specific for p65, phospho-p65, phospho-IKKalpha/beta, IKKalpha, pSTAT1 (tyr701), pSTAT3 (tyr705; Cell Signaling), pSTAT6 (tyr641), total STAT3, BCL-XL, and actin (Cell Signaling) and MCL1, BCL-2 (Santa Cruz Biotechnology). For enhanced chemiluminescence (ECL)-based detection, Western blots are developed with ECL plus Western blot system (Santa Cruz Biotechnology) and the secondary horseradish peroxidase-conjugated antibodies goat anti-rabbit IgG or goat anti-mouse IgG are used (Santa Cruz Biotechnology). Western blots are quantified by the ImageJ software (version 1.6.0).

### Assessment of Intracellular ROS/Superoxide Levels

Intracellular ROS/superoxide levels were assessed by the ROS/superoxide Detection Assay Kit (ab139476, Abcam, Cambridge, UK) according to the manufacturer's protocol. After washing in serum-free medium, cells were diluted to desired density (1.11×10⁵/ml).

Cells were incubated with OU749 for 10 minutes prior to addition of serum or albumin (FCS or HS, both 10%, BSA or different HSA preparations as indicated), or albumin-supplemented FCS (supplementation with HSA or BSA: final albumin concentration 4-5 mg/ml). The final cell concentration was 1×10⁵/ml/tube (volume 1 ml). Mean fluorescence intensities of at least 10000 events were assessed and compared.

### Assessment of the Total Anti-Oxidant Capacity of Serum and Albumins, and Albumin-Supplemented FCS

The total anti-oxidant capacity of serum and albumins, and albumin-supplemented FCS was measured using the Cayman Chemical Antioxidant Assay Kit™ (Cayman Chemical, Ann Arbor, Mich., USA) according to the manufacturer's instructions. In brief, the anti-oxidant assay relies on the ability of anti-oxidants in the sample to inhibit the oxidation of ABTS® (2,2′-azino-di-[3-ethylbenzthiazoline sulphonate]). The capacity of the anti-oxidants in the sample to prevent ABTS oxidation is compared with that of Trolox®, a water-soluble tocopherol analogue, and is quantified as molar Trolox® equivalents.

### Assessment of NF-κB Family Member Activation Levels

After 4 h incubation with defined concentrations of inhibitors of the NF-κB signalling pathway with or without co-incubation with OU749, nuclear extracts were prepared using the AM nuclear extract kit (Active Motif, La Hulpe, Belgium) according to the manufacturer's protocol. Protein concentration of the respective extracts was measured according to the Bradford method and 10 μg of total protein per preparation was used for assessment of the NF-κB family member activation.

For the assessment of NF-κB family member activation (p65, p50, c-Rel, p52 and RelB) the TransAM® NF-κB Family Transcription Factor Assay kit was applied (Active Motif, La Hulpe, Belgium) according to the manufacturer's instructions. Results were expressed as relative activation levels (relative to the activation of DMSO controls either in the presence of 10% FCS or 10% HS) and compared between different experimental settings.

The invention is further described by the following non-limiting Examples.

### Reference Example 1

### Clinical Administration of Leflunomide in Concentrations Sufficient to Achieve Effect In Vivo—Negative Result.

Leflunomide was given to two refractory CLL patients Patient No. 1: male, 70 yr; CLL first diagnosis 1999, cytogenetics: del17p; del13q; multiple previous treatments (CHOP, DexaBEAM, fludarabine, alemtuzumab). Patient No. 2: male, 76 yr; CLL first diagnosis 1998, cytogenetics: del13q14, del17p, TP53-mut, multiple previous treatments (chlorambucil, fludarabine, rituximab, ABVD, alemtuzumab).

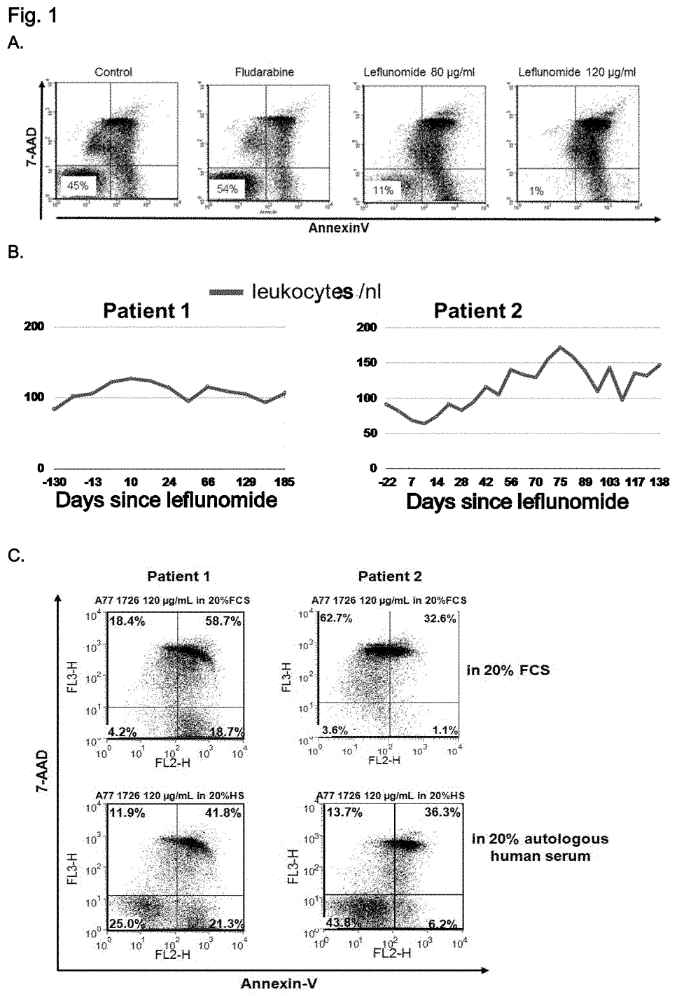

The dosage was 20 mg p.o. daily. Plasma concentrations of the active metabolite of leflunomide, A77 726, of about 176 μg/ml, i.e. about 635 μM, are achievable. Serum concentrations were measured in steady-state by HPLC and were found to be >100 μg/ml (i.e. >370 μM). NF-κB inhibitor activity could be assessed by apoptosis assays employing FCS in vitro. However, in vivo no reduction of peripheral leukocyte counts could be achieved in patients after leflunomide treatment for up to 180 days. In-vitro testing of leflunomide treatment of primary patient cells employing FCS versus autologous HS revealed protective effects of HS (i.e. diminished efficacy of leflunomide in HS as compared to FCS). Results of clinical administration of leflunomide are summarised in FIG. 1.

### Example 2

In Vitro Efficacy of NF-κB Inhibitor is Lost when FCS Component of Culturing Medium is Replaced with HS in MEC1, MOLM13 and HL60 Cells.

After washing in serum-free medium cells were diluted to desired density (3.70×10⁵/ml for MEC1 and MOLM13, 1.25×10⁵/ml for HL60) and HS and FCS was added both in a final concentration of 10%. Then 90-μl aliquots of the cell suspension (densities after addition of serum: 3.33×10⁵/ml for MEC1 and MOLM13, 1.1×10⁵/ml for HL60) were dispensed into 96-well flat-bottomed microtitre plates containing 10 μl/well of serial drug dilutions of NF-κB inhibitors. The cell concentrations in the 96 well plate were: 3×10⁴/well for MEC1 and MOLM13, 1×10⁴/well for HL60. The dose ranges tested are summarised in Table 1.

TABLE 1
Concentration ranges of NF-κB inhibitors (μM).

| | MEC1 | MOLM13 | HL60 |
|---|---|---|---|
| Bay 11-7082 | 1.25-10 | 1.25-10 | 1.25-10 |
| EF24 | 1.25-5 | 1.25-5 | 1.25-5 |
| Leflunomide | 25-200 | 25-200 | 12.5-100 |
| Sorafenib | 1.25-20 | 0.01-0.16 | 1.25-20 |

Since inhibitors were initially dissolved in DMSO, serial drug dilutions and controls were prepared to finally achieve 0.2% DMSO in all wells (total volume 100 μl); no anti-proliferative effects could be observed at this DMSO concentration in the investigated cell lines.

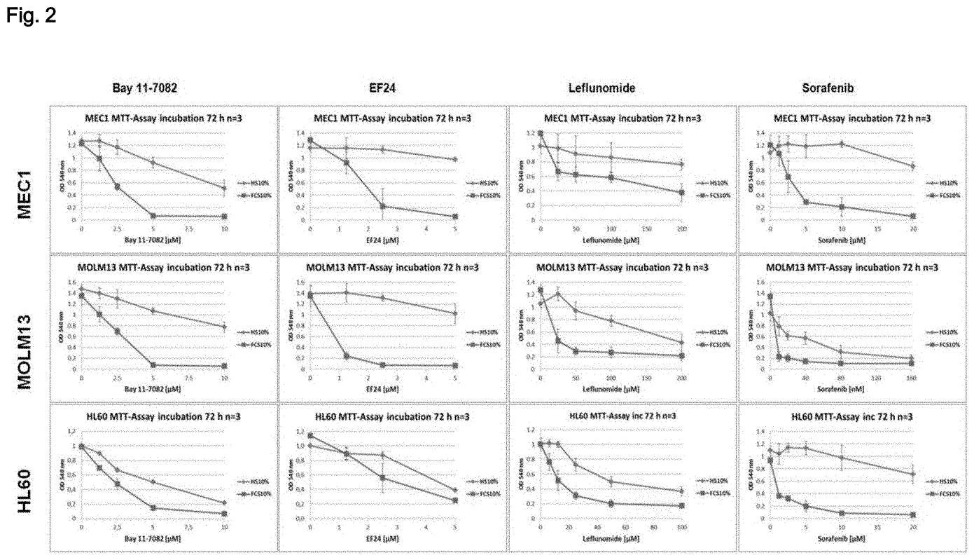

The results show that the activity of NF-κB inhibitors in cell systems supplemented with HS is strongly diminished as compared to cell systems supplemented with FCS, i.e. HS exerts protective effects in the context of NF-κB inhibitors in vitro. Effects on cell growth of BAY 11-7082, EF24, leflunomide, and sorafenib in the CLL cell line MEC1 and the AML cell lines MOLM13 and HL60 in HS versus FCS supplemented cell systems are given in FIG. 2. The corresponding IC50 values are summarised in Table 2.

TABLE 2
Effects of BAY 11-7082, EF24, leflunomide, and sorafenib on the proliferation of MEC1,
MOLM13 and HL60 cells in HS versus FCS supplemented cell systems after 72 h treatment (IC50 values in μM).

| | MEC1 | | | MOLM13 | | | HL60 | | |
|---|---|---|---|---|---|---|---|---|---|
| | HS 10% | FCS 10% | P | HS 10% | FCS 10% | P | HS 10% | FCS 10 % | P |
| Bay 11-7082 | 9.15 ± 1.73 | 1.85 ± 0.04 | ** | 12.61 ± 3.51 | 2.04 ± 0.20 | ** | 5.04 ± 0.32 | 2.47 ± 0.30 | * |
| EF24 | 13.82 ± 4.93 | 1.66 ± 0.44 | * | 8.53 ± 0.30 | 0.30 ± 0.03 | *** | 4.27 ± 0.10 | 1.75 ± 0.33 | ** |
| Leflunomide | 586.5 ± 29.8 | 72.52 ± 5.12 | *** | 175.0 ± 48.7 | 14.79 ± 3.55 | * | 77.07 ± 2.91 | 16.31 ± 1.12 | *** |
| Sorafenib | 34.23 | 3.03 | ** | 0.037 ± 0.00 | 0.002 ± 0.0 | ** | 35.21 ± 10. | 1.44 ± 0. | ** |

FCS: fetal calf serum; HS: human serum; *P < 0.05; **P < 0.01; ***P < 0.001 (t test)

### Example 3

In Vitro Efficacy of NF-κB Inhibitors is Restored in MEC1, MOLM13 and HL60 Cells when γ-GT Inhibitor is Added to a Cell Culture Containing HS.

After washing in serum-free medium cells were diluted to desired density (3.70×10⁵/ml for MEC1 and MOLM13, 1.25×10⁵/ml for HL60), cells were pre-incubated with OU749 in a concentration of 200 μM for 10 minutes (this concentration was found to exert no toxic effects in HS-supplemented systems) or DMSO control, and HS was added in a final concentration of 10%. Then 90-μl aliquots of the cell suspension (densities after addition of serum: 3.33×10⁵/ml for MEC1 and MOLM13, 1.1×10⁵/ml for HL60) were dispensed into 96-well flat-bottomed microtitre plates containing 10 μl/well of serial drug dilutions of NF-κB inhibitors. The cell concentrations in the 96 well plates were: 3×10⁴/well for MEC1 and MOLM13, 1×10⁴/well for HL60. The dose ranges tested are summarised in Table 1.

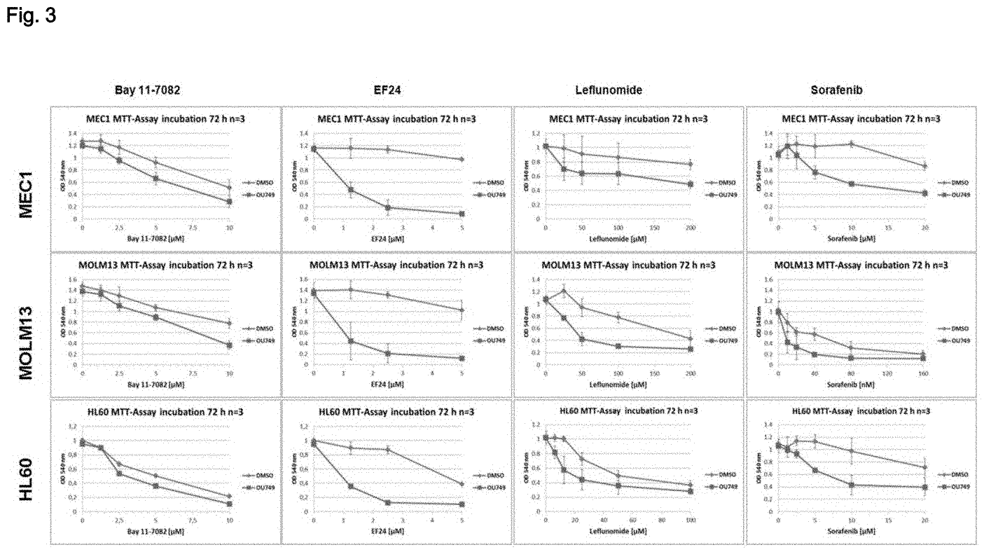

The results show that addition of non-toxic doses of the γ-glutamyltransferase inhibitor OU749 (200 μM) are able to enhance/restore the activity of NF-κB inhibitors in HS supplemented cell systems/n vitro. Effects on cell growth of BAY 11-7082, EF24, leflunomide, and sorafenib in the CLL cell line MEC1 and the AML cell lines MOLM13 and HL60 in HS after addition of 200 μM OU749 versus DMSO controls are shown in FIG. 3. The corresponding IC50 values are summarised in Table 3.

TABLE 3
Effects of BAY 11-7082, EF24, leflunomide, and sorafenib on the proliferation of MEC1,
MOLM13 and HL60 cells in HS after addition of 200 μM OU749 versus DMSO control (72 h treatment, IC50 values in μM).

| | MEC1 | | | MOLM13 | | | HL60 | | |
|---|---|---|---|---|---|---|---|---|---|
| | DMSO | OU749 | P | DMSO | OU749 | P | DMSO | OU749 | P |
| Bay11-7082 | 9.15 ± 1.73 | 4.64 ± 0.41 | * | 12.61 ± 3.51 | 5.68 ± 0.08 | * | 5.04 ± 0.32 | 2.90 ± 0.16 | * |
| EF24 | 13.82 ± 4.93 | 1.06 ± 0.25 | * | 8.53 ± 0.30 | 0.84 ± 0.68 | *** | 4.27 ± 0.10 | 0.67 ± 0.15 | ** |
| Leflunomide | 586.5 ± 29.8 | 176.2 ± 22.1 | *** | 175.0 ± 48.7 | 50.2 ± 2.0 | * | 77.07 ± 2.91 | 20.33 ± 4.53 | *** |
| Sorafenib | 34.23 ± 2.9 | 12.74 ± 1.18 | *** | 0.037 ± 0.00001 | 0.003 ± 0.0009 | *** | 35.21 ± 10.29 | 9.65 ± 1.06 | * |

OU749 = 200 μM; *P < 0.05; **P < 0.01; ***P < 0.001 (t test)

### Example 4

In Vitro Efficacy of NF-κB Inhibitors is Restored in MEC1, MOLM13 and HL60 Cells when Glutathione Catabolism Inhibitors are Added to a Cell Culture Containing HS.

After washing in serum-free medium cells were diluted to desired density (3.70×10⁵/ml), cells were pre-incubated with auranofin (single agent: 0, 1, 2 μM), BSO (single agent: 0, 100, 500 μM) or combinations (auranofin: 0, 0.25, 0.5 μM; BSO: 1.25-10 μM) or DMSO control for 10 minutes, and HS was added in a final concentration of 10%. Then 90-μl aliquots of the cell suspension (densities after addition of serum: 3.33×10⁵/ml) were dispensed into 96-well flat-bottomed microtitre plates containing 10 μl/well of serial drug dilutions of NF-κB inhibitors. The cell concentrations in the 96 well plate were: 3×10⁴/well. The tested dose ranges of BAY 11-7082 and EF24 are summarised in Table 1 above.

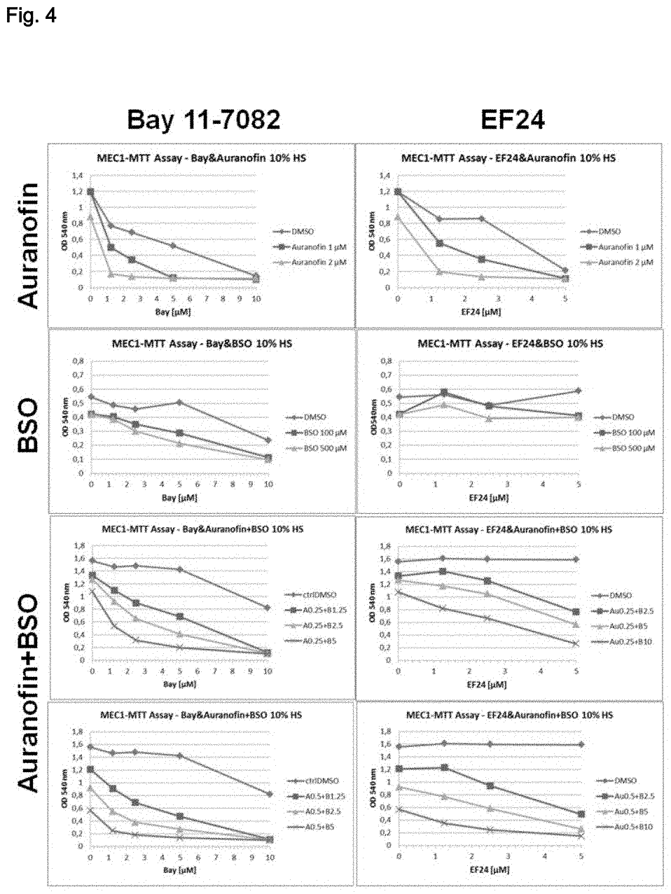

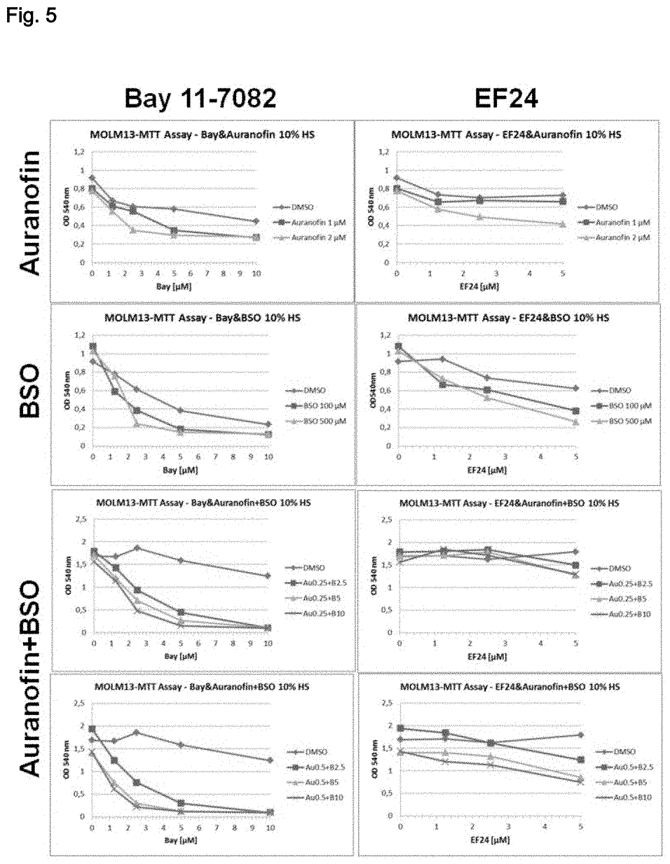

The results show that addition of low-dose combinations of the glutathione catabolism inhibitors auranofin and BSO was able to enhance/restore the activity of NF-κB inhibitors in HS supplemented cell systems in vitro. Effects on cell growth of BAY 11-7082 and EF24 in the CLL cell line MEC1 and the AMLcell line MOLM13 after addition of auranofin and BSO versus control are illustrated by the representative experiment shown in FIG. 4 and FIG. 5, respectively.

### Example 5

### Glutathione Catabolism has Direct Influence on Activity of NF-κB Inhibitors.

After washing in serum-free medium cells were diluted to desired density (3.70×10⁵/ml), cells were pre-incubated with cysteinyl-glycin (Cys-Gly: 0, 40, 400, 4000 μM for experiments with FCS or 0, 10, 100, 1000 μM for experiments with HS) for 10 minutes, and FCS and HS was added both in a final concentration of 10%. Then 90-μl aliquots of the cell suspension (densities after addition of serum: 3.33×10⁵/ml) were dispensed into 96-well flat-bottomed microtitre plates containing 10 μl/well of serial drug dilutions of NF-κB inhibitors. The cell concentrations in the 96 well plate were: 3×10⁴/well. The tested dose ranges of BAY 11-7082 are summarised in Table 1 above.

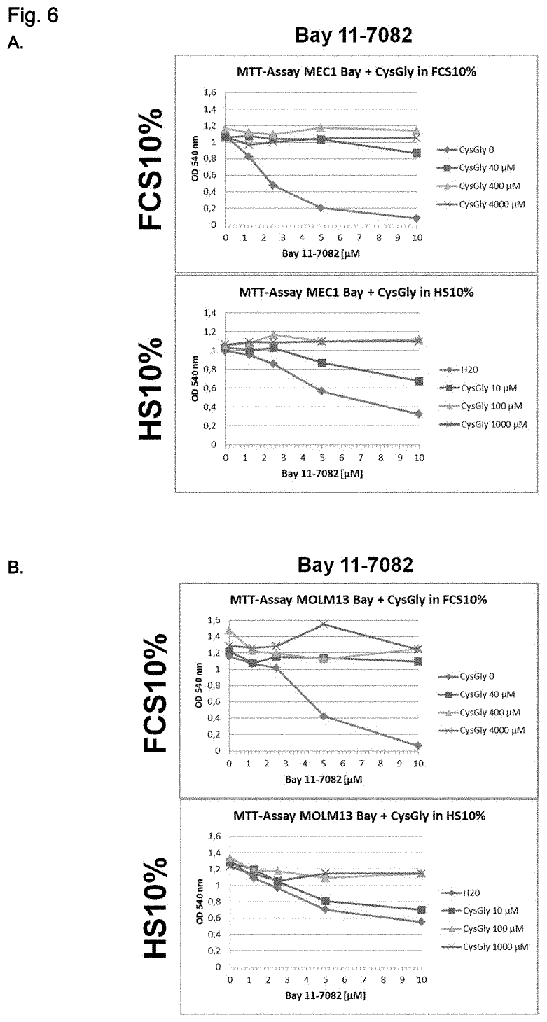

The results show that addition of cysteinyl-glycin was able to decrease the activity of NF-κB inhibitors in FCS (and to a lesser extent in HS) supplemented cell systems in vitro. Effects on cell growth of BAY 11-7082 in the CLL cell line MEC1 and the AMLcell line MOLM13 in the presence of cysteinyl-glycin versus H₂O are illustrated by the representative experiment shown in FIG. 6.

### Example 6

Reduced ROS Levels Detected in MEC1 and MOLM13 Cells when Cultured in HS-Containing Cell Culture Medium Compared to the Levels Detectable when Cultured in FCS-Containing Cell Culture Medium.

ROS and superoxide levels were assessed by flow cytometry using the cellular ROS/superoxide Detection Assay Kit (ab139476, Abcam, Cambridge, UK) according to the manufacturer's protocol. After washing in serum-free medium, cells were diluted to desired density (1.11×10⁵/ml), and FCS and HS was added both in a final concentration of 10% (final cell concentration 1×10⁵/ml/tube, volume 1 ml). Mean fluorescence intensities of at least 10000 events were assessed and compared.

ROS levels in MEC1 and MOLM13 cells were significantly reduced when cultured in HS-containing cell culture medium as compared to the levels detectable when cells were cultured in FCS-containing cell culture medium. The results of ROS/superoxide measurements in the CLL cell line MEC1 and the AMLcell line MOLM13 in FCS versus HS supplemented medium are given in FIG. 6.

### Example 7

Increased ROS Levels Detected in MEC1 and MOLM13 Cells when Cultured in HS-Containing Cell Culture Medium and Exposed to a γ-GT Inhibitor.

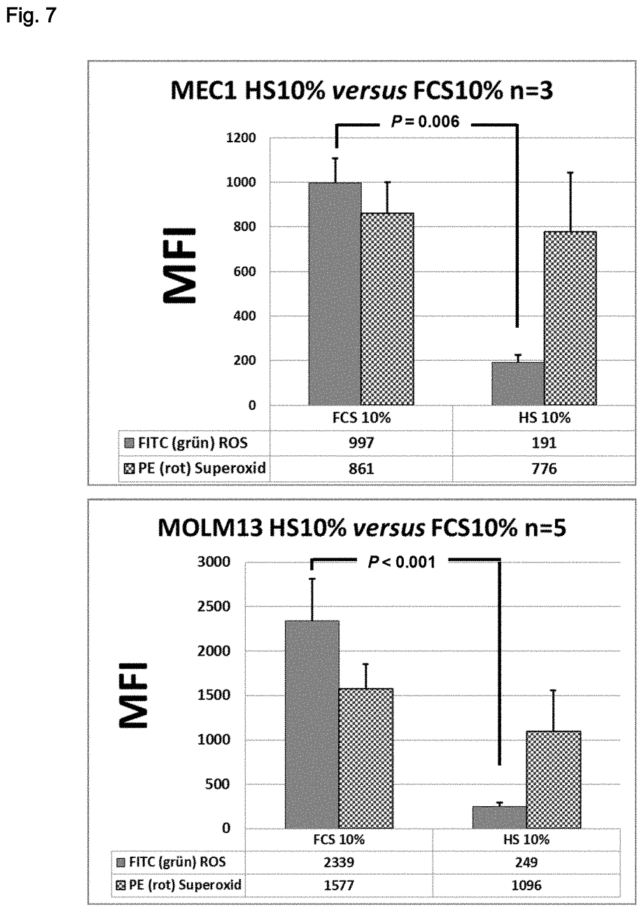

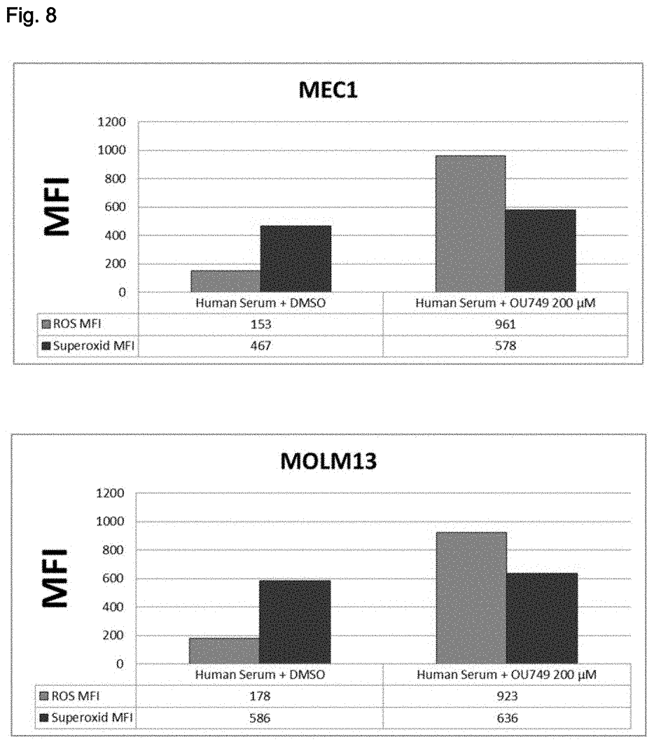

ROS and superoxide levels were assessed by flow cytometry using the cellular ROS/superoxide Detection Assay Kit (ab139476, Abcam, Cambridge, UK) according to the manufacturer's protocol. After washing in serum-free medium and dilution to the desired density (1.11×10⁵/ml), cells were pre-incubated with OU749 in a concentration of 200 μM or DMSO control for 10 minutes (this concentration was found to exert no toxic effects in HS-supplemented systems), and HS was added in a final concentration of 10% (final cell concentration 1×10⁵/ml/tube, volume 1 ml). Mean fluorescence intensities of at least 10000 events were assessed and compared. Addition of OU749 enhanced ROS levels in MEC1 and MOLM13 cells when cultured in HS-containing cell culture medium. The results of ROS/superoxide measurements in OU749 versus DMSO control treated MEC1 and MOLM13 cells are illustrated by the representative experiment shown in FIG. 7.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### Example 8

### Total Anti-Oxidant Capacity of Different Sera and Albumins and Albumin-Supplemented Fetal Calf Serum

The total anti-oxidant capacity of HS or pooled HS is significantly higher as compared to standard FCS. Similarly, the total anti-oxidant capacity of BSA and HAS as well as of BSA-or HSA-supplemented FCS is also significantly higher than that of FCS.

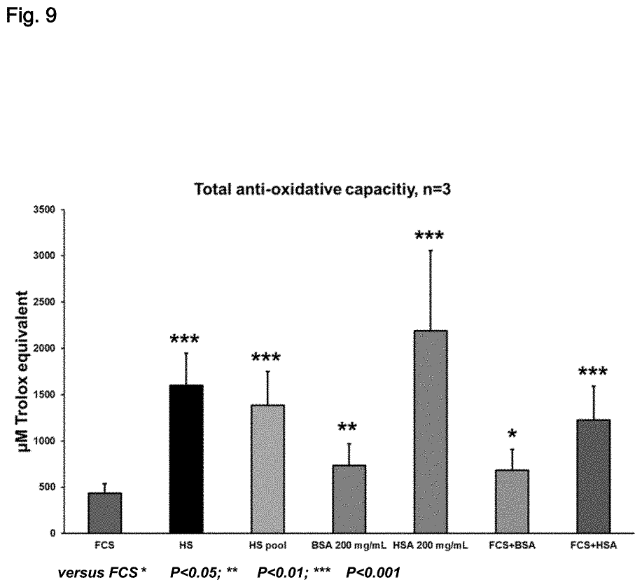

FCS was supplemented with BSA or HSA to achieve albumin concentrations of −40 mg/ml. The total anti-oxidant capacity (quantified as molar Trolox® equivalents)±standard deviation is depicted (n=3) in FIG. 9. Based on the Two-sided t test the relevant p-values were determined and are indicated in FIG. 9 as follows: *P<0.05; **P<0.01; ***P<0.001.

### Example 9

Levels of Intracellular Reactive Oxygen Species (ROS) Depend on the Extracellular Environment (i.e. Serum-Free, Serum- or Albumin-Supplemented Cell Culture Medium)

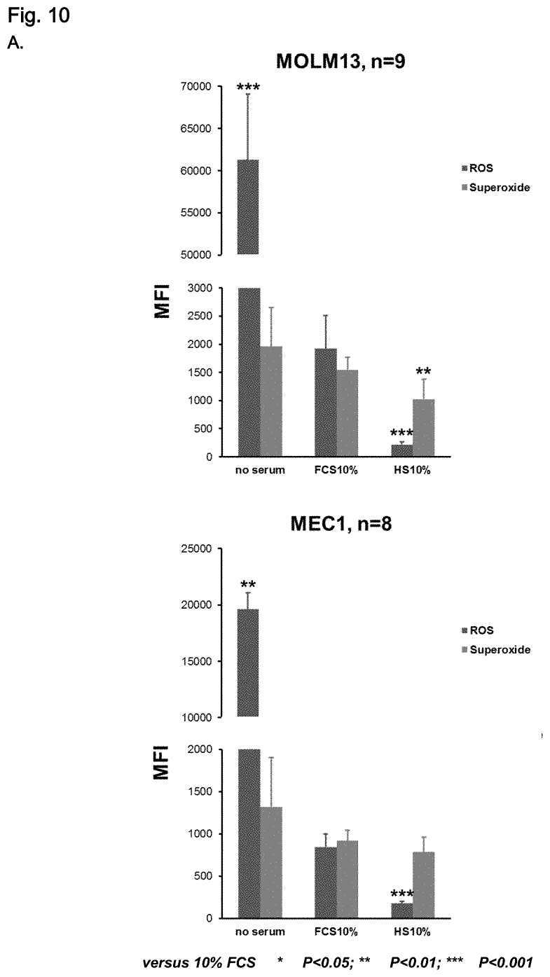

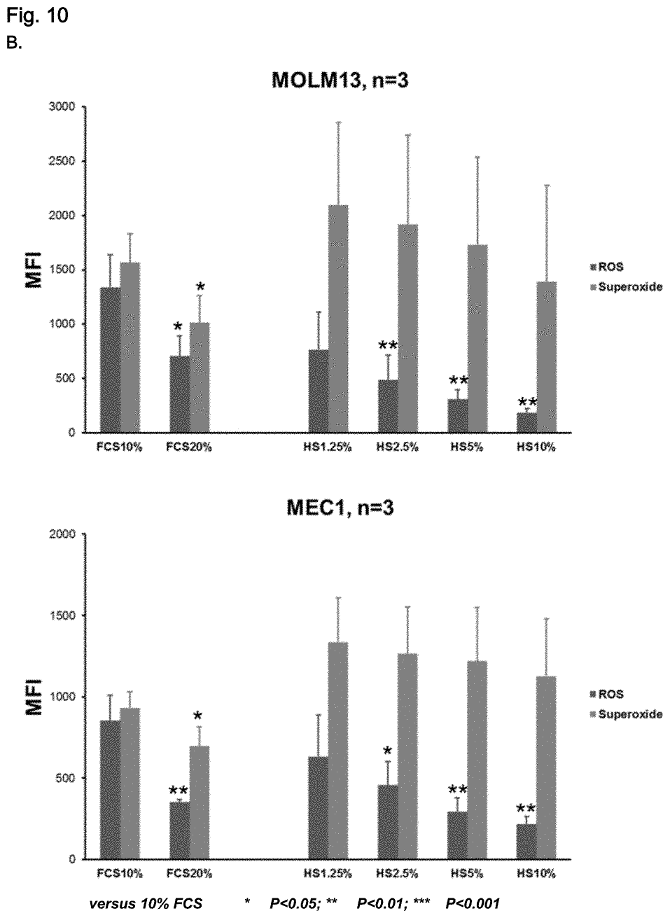

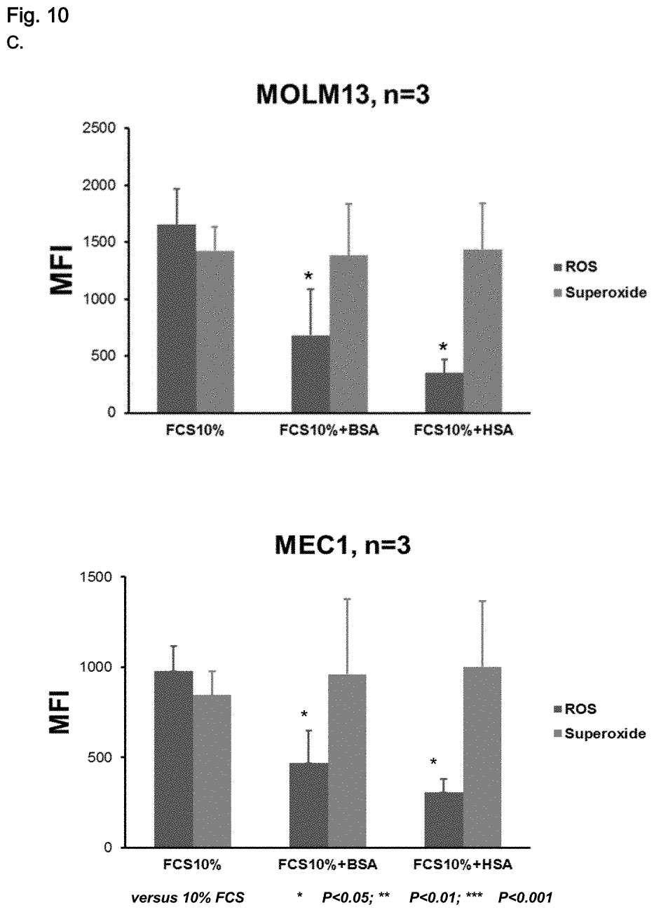

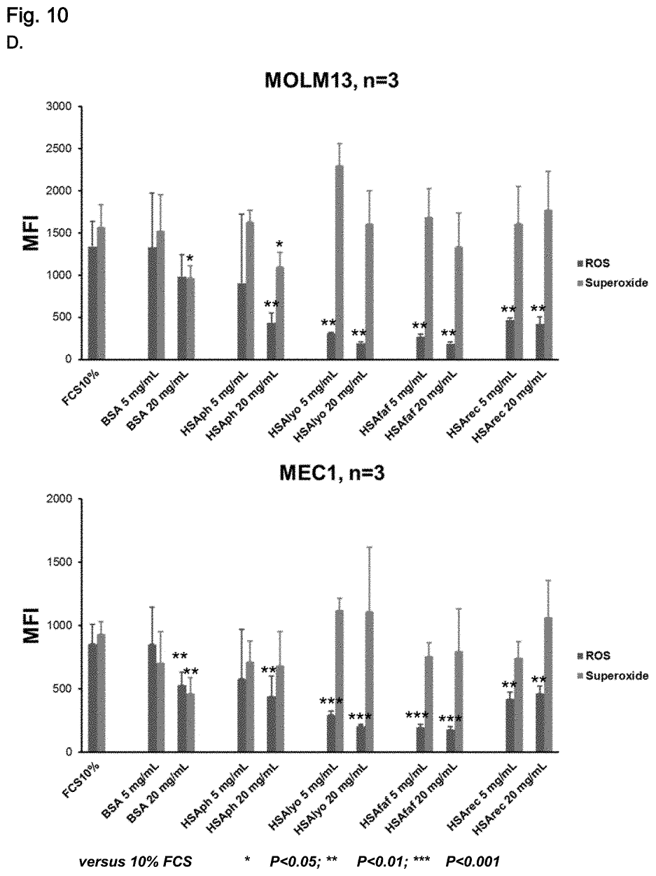

As shown in FIG. 10A, when compared with cells grown in FCS-supplemented cell culture medium (10% FCS), both MOLM13 and MEC1 cells show significantly higher intracellular ROS levels in serum-free conditions. Further, the presence of human serum (10% HS) significantly reduces intracellular ROS levels in both cell lines.

As shown in FIG. 10B both in MOLM13 and MEC1 cells, step-wise increases in the concentration of serum supplements (both FCS and HS) results in dose-dependent step-wise reduction of intracellular ROS levels.

Similarly, FIG. 10C shows that supplementation of FCS with BSA or HSA results in reduced intracellular ROS levels in MOLM13 and MEC1 cells. FCS was supplemented with BSA or HSA to achieve albumin concentrations of ˜4 mg/ml per tube.

When comparing the intracellular ROS-levels of both MOLM13 and MEC1 cells cultured in 10% FCS-supplemented cell culture medium with those of MOLM13 and MEC1 cells cultured in 10% FCS-supplemented cell culture medium further containing different albumin preparations, it becomes apparent that the intracellular ROS levels decrease in a dose-dependent fashion inversely correlated with increasing amounts of albumin. The various albumin preparations tested (as shown in FIG. 10D) are: BSA; HSAph—pharmaceutical-grade HSA; HSAfaf—fatty-acid-free HSA; HSAlyo—commercial lyophilized HSA; HSArec-recombinant HSA.

Intracellular ROS and superoxide levels were quantified as mean fluorescence intensity [MFI])±standard deviation as depicted in FIG. 10D. Based on the Two-sided t test the relevant p-values were determined and are indicated in FIG. 10 as follows: *P<0.05; **P<0.01; ***P<0.001.

From the above, it is apparent that the levels of intracellular reactive oxygen species (ROS) depend on the extracellular environment (i.e. serum-free, serum- or albumin-supplemented cell culture medium).

### Example 10

In Vitro Effects of NF-κB Inhibitors on Cell Growth (Expressed as IC₅₀ Concentrations in MEC1 and MOLM13 Cells after 72 h Treatment) Depend on the Concentration of Serum or Albumin in the Cell Culturing Medium

After washing in serum-free medium cells were diluted to desired density (3.70×10⁵/ml for MEC1 and MOLM13) and HS, FCS or albumin-supplemented FCS was added in the respective final concentrations. FCS was supplemented with BSA or HSA to achieve albumin concentrations of ˜4 mg/ml per well (FCS+BSAlow and FCS+HSAlow) or to achieve albumin concentrations of ˜7 mg/ml per well (FCS+BSAhigh and FCS+HSAhigh).

Then 90-μl aliquots of cell suspension (densities after addition of serum: 3.33×10⁵/ml for MEC1 and MOLM13) were dispensed into 96-well flat-bottomed microtitre plates containing 10 μl/well of serial drug dilutions of NF-κB inhibitors. The cell concentrations in the 96 well plate were: 3×10⁴/well for MEC1 and MOLM13. The dose ranges tested are summarized in Table 4.

TABLE 4
Concentration ranges of NF-κB inhibitors (μM).

| | MOLM13 | MEC1 |
|---|---|---|
| Bay 11-7082 | 0.3125-20 | 0.3125-20 |
| EF24 | 0.3125-20 | 0.3125-20 |
| Leflunomide (A77 1726) | 6.25-400 | 6.25-400 |
| Sorafenib | 0.005-0.320 | 0.625-40 |

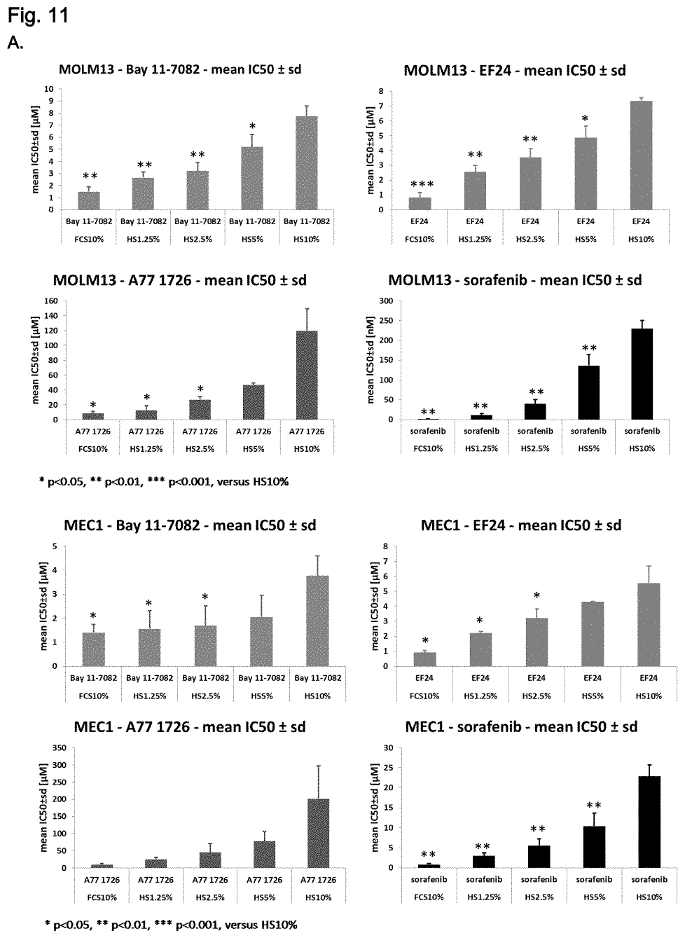

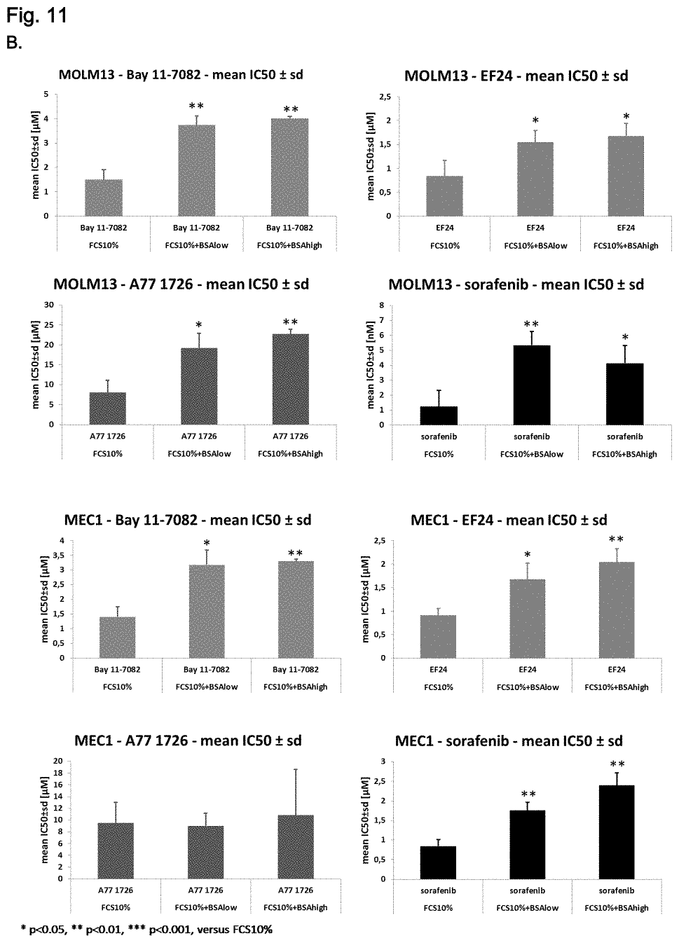

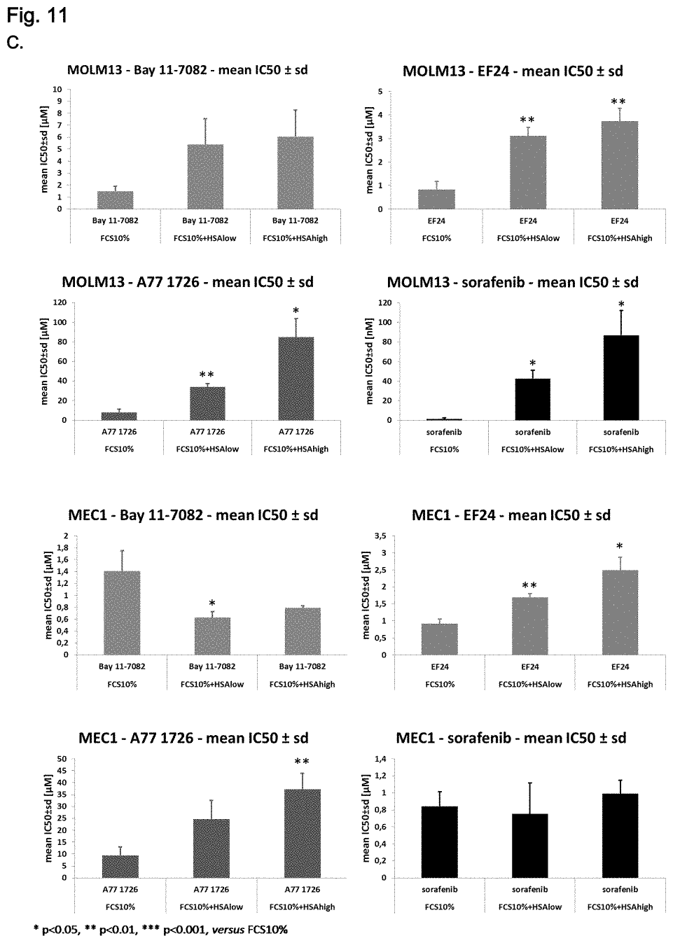

Compared to FCS-supplemented cell culture medium (10% FCS), in both MOLM13 and MEC1 cell lines the sensitivity of NF-κB inhibitors decreased depending on the concentration of human serum present in the cell culture medium (FIG. 11A).

Compared to FCS-supplemented cell culture medium (10% FCS), in both MOLM13 and MEC1 cell lines the sensitivity of NF-κB inhibitors was also lower when 10% FCS was supplemented with BSA (FIG. 11B).

Similar effects were also observed when 10% FCS was supplemented with HSA (FIG. 11C).

In FIG. 11, IC₅₀ concentrations±standard deviation of the respective NF-κB inhibitors are depicted (n=3). Based on the Two-sided t test the relevant p-values were determined and are indicated in FIG. 11 as follows: *P<0.05; **P<0.01; ***P<0.001 the *P<0.05; **P<0.01; ***P<0.001.

From the above it is apparent that the in vitro effects of NF-κB inhibitors on cell growth (expressed as IC₅₀ concentrations in MEC1 and MOLM13 cells after 72 h treatment) depend on the concentration of serum or albumin in the cell culturing medium.

### Example 11

In Vitro Effects of the γ-Glutamyltransferase (γ-GT) Inhibitor OU749 on the Cell Growth of MOLM13 and MEC1 Cells after 72 h Treatment in FCS- Versus HS-Supplemented Cell Culturing medium

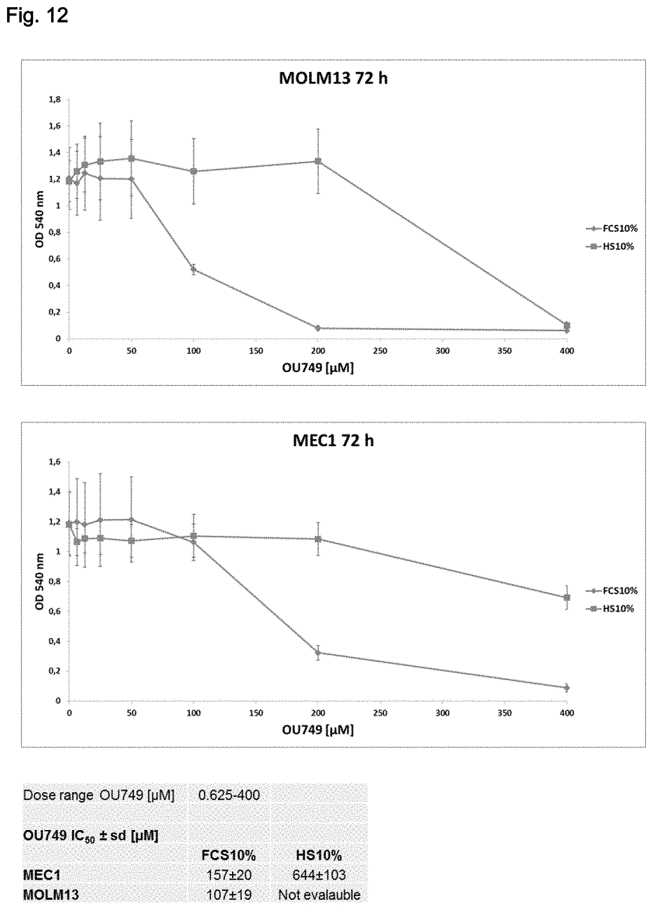

The γ-GT inhibitor OU749 does not exert anti-proliferative effects on either MOLM13 or MEC1 cells cultured in 10% HS-supplemented culture medium, unless the OU749 is increased to concentrations of 200 μM or higher. In contrast, in 10% FCS-supplemented cell culture after 72 h, anti-proliferative effects of OU749 are already evident at much lower OU749 concentrations. In particular, anti-proliferative effects are apparent from concentrations above 50 μM in both cell lines (FIG. 12).

As such, the in vitro effects of the γ-glutamyltransferase (γ-GT) inhibitor OU749 on the cell growth of MOLM13 and MEC1 cells after 72 h treatment in FCS- versus HS-supplemented cell culturing medium.

### Example 12

In Vitro Effects of NF-κB Inhibitors on Cell Growth Expressed as IC₅₀ Concentrations in MOLM13 and MEC1 Cells after 72 h Treatment are Enhanced in MOLM13 and MEC1 Cells when γ-GT Inhibitor is Added to a Cell Culture Containing FCS and Restored when γ-G T Inhibitor is Added to a Cell Culture Containing HS or Albumin-Supplemented FCS

10% FCS was supplemented with bovine serum albumin (BSA) or human serum albumin (HSA) to achieve albumin concentrations of ˜4 mg/ml per well (FCS+BSAlow and FCS+HSAlow) or to achieve albumin concentrations of ˜7 mg/ml per well (FCS+BSAhigh and FCS+HSAhigh). OU749 was added to the respective culture conditions in increasing concentration (25, 50 and 100 μM for FCS; 50, 100 and 200 μM for HS or albumin-supplemented FCS).

For dose ranges of NF-κB inhibitors tested see Table 4.

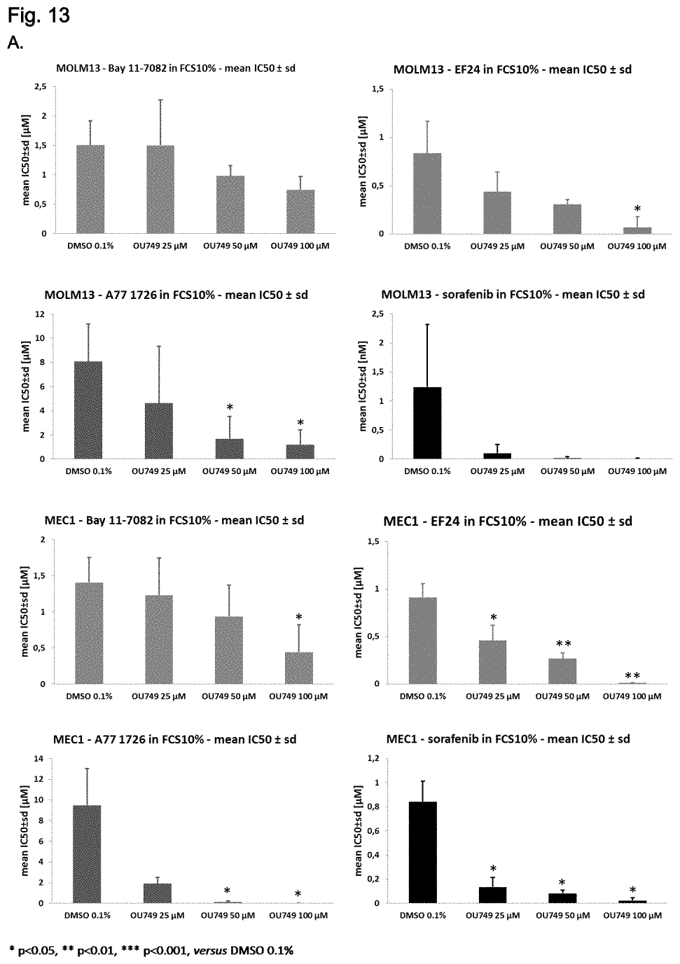

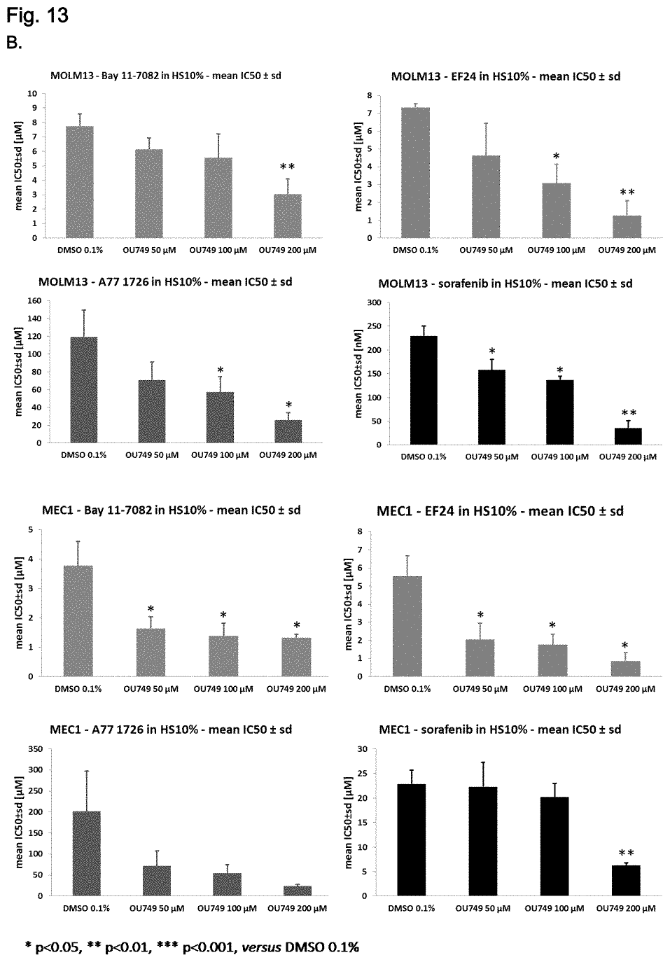

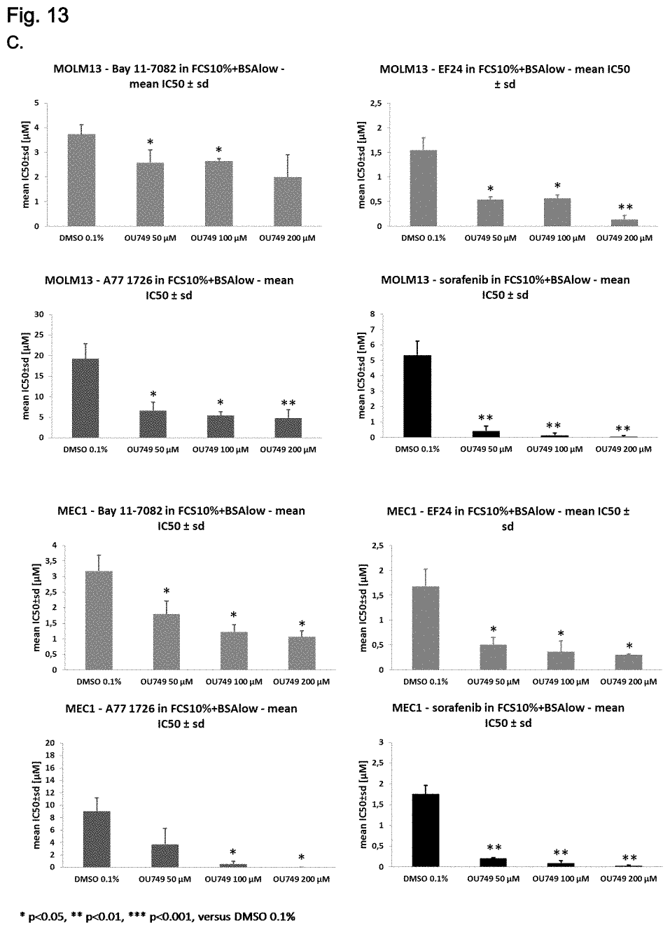

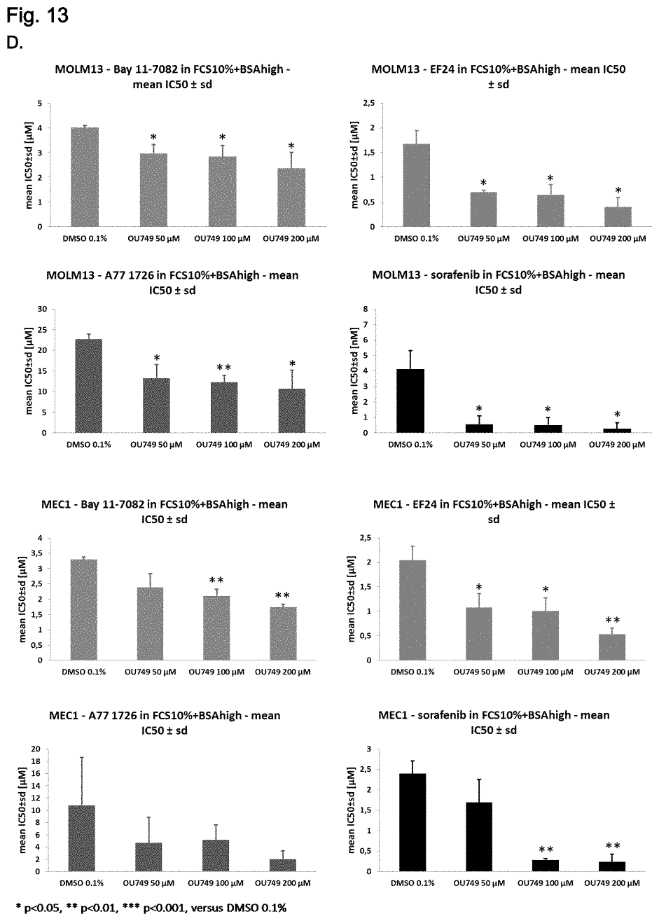

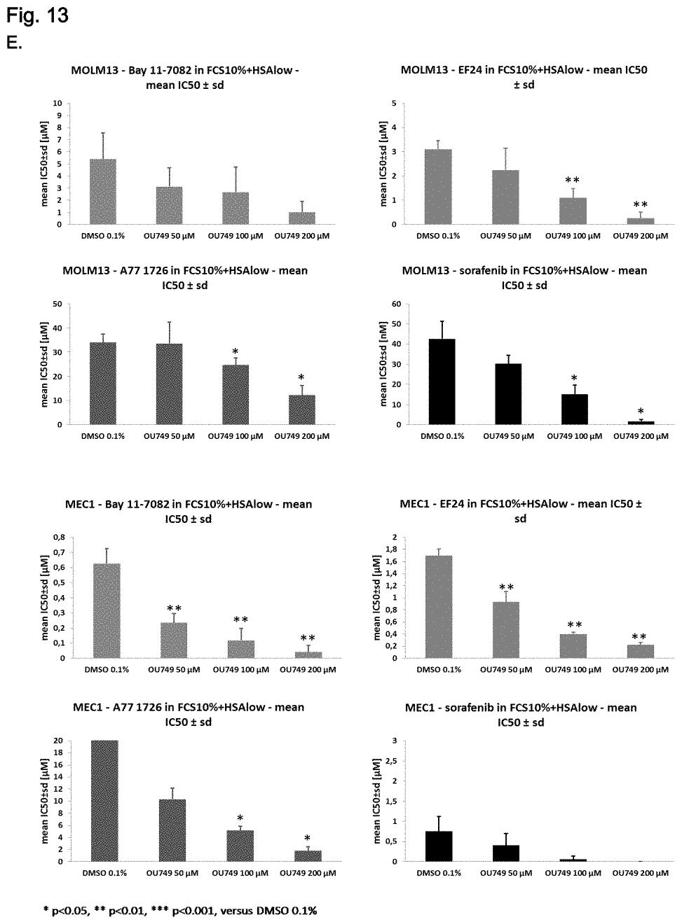

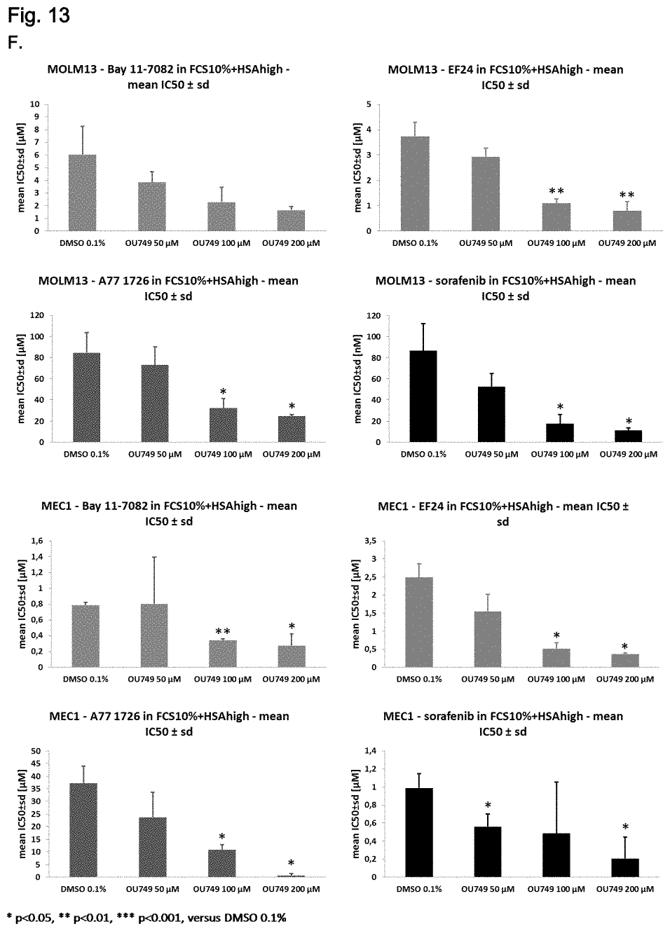

As shown in FIG. 13A, anti-proliferative activity of NF-κB inhibitors in MOLM13 and MEC1 cells is enhanced when OU749 is added to a cell culture containing 10% FCS.

As shown in FIG. 13B, anti-proliferative activity of NF-κB inhibitors in MOLM13 and MEC1 cells is enhanced/restored when OU749 is added to a cell culture containing 10% HS.

As shown in FIG. 13C, anti-proliferative activity of NF-κB inhibitors in MOLM13 and MEC1 cells is enhanced/restored when OU749 is added to a cell culture containing 10% FCS supplemented with lower doses of BSA (FCS+BSAlow, albumin concentrations ˜4 mg/ml per well).

As shown in FIG. 13D, anti-proliferative activity of NF-κB inhibitors in MOLM13 and MEC1 cells is enhanced/restored when OU749 is added to a cell culture containing 10% FCS supplemented with higher doses of BSA (FCS+BSAhigh, albumin concentrations ˜7 mg/ml per well).

As shown in FIG. 13E, anti-proliferative activity of NF-κB inhibitors in MOLM13 and MEC1 cells is enhanced/restored when OU749 is added to a cell culture containing 10% FCS supplemented with lower doses of HSA (FCS+HSAlow, albumin concentrations ˜4 mg/ml per well).

As shown in FIG. 13F, anti-proliferative activity of NF-κB inhibitors in MOLM13 and MEC1 cells is enhanced/restored when OU749 is added to a cell culture containing 10% FCS supplemented with higher doses of HSA (FCS+HSAhigh, albumin concentrations ˜7 mg/ml per well).

IC₅₀ concentrations±standard deviation of the respective NF-κB inhibitors are depicted (n=3). Based on the Two-sided t test the relevant p-values were determined and are indicated in FIG. 13 as follows: *P<0.05; **P<0.01; ***P<0.001.

Accordingly, it is apparent that the in vitro effects of NF-κB inhibitors on cell growth expressed as IC₅₀ concentrations in MOLM13 and MEC1 cells after 72 h treatment are enhanced in MOLM13 and MEC1 cells when γ-GT inhibitor is added to a cell culture containing FCS and restored when γ-GT inhibitor is added to a cell culture containing HS or albumin-supplemented FCS.

### Example 13

Treatment of MOLM13 and MEC1 with γ-GT Inhibitor Increases the Levels of Intracellular Reactive Oxygen Species (ROS) in Cell Culture Systems Containing Serum (10% FCS and 10% HS) or Albumin (BSA and HSA 20 mg/ml)

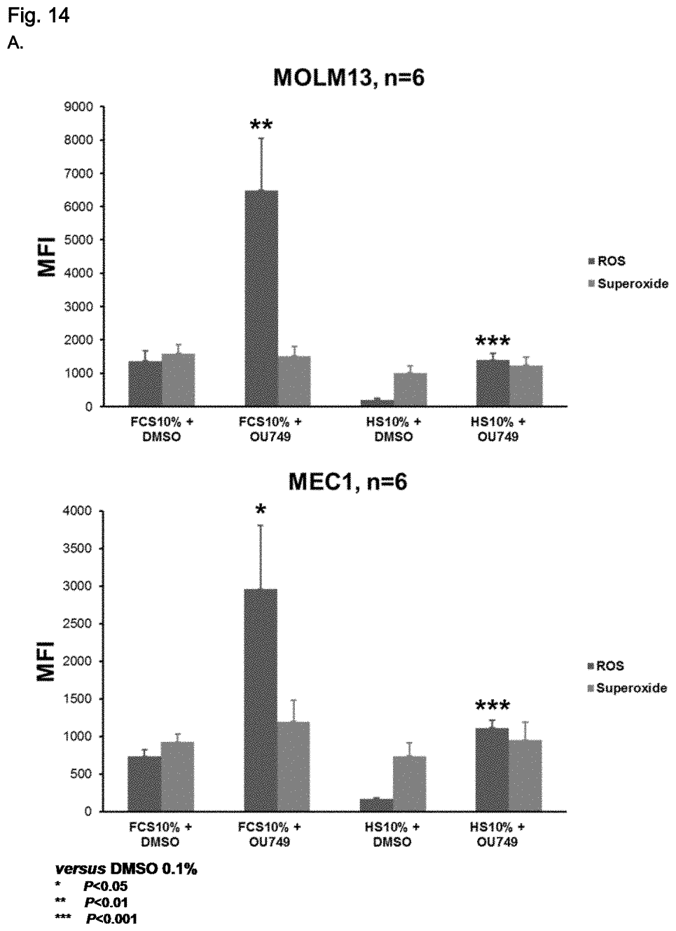

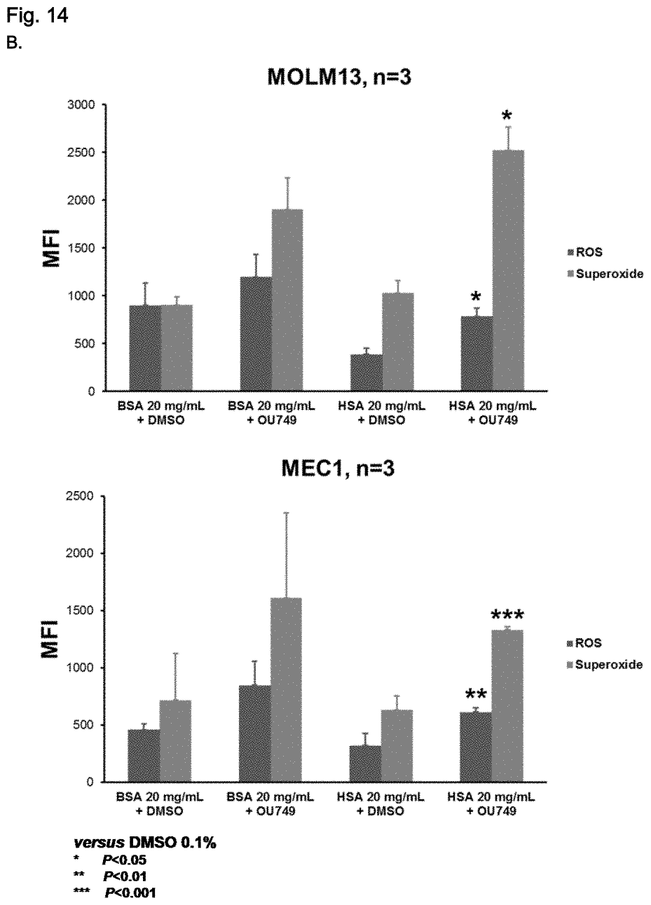

As shown in FIG. 14A, compared to DMSO controls, in both MOLM13 and MEC1 cells, treatment with γ-GT inhibitor significantly increases intracellular ROS levels in both FCS- and HS-supplemented cell culture systems.

Similarly, in both MOLM13 and MEC1 cells, treatment with γ-GT inhibitor significantly increases intracellular ROS levels in both BSA or HSA supplemented cell culture systems as compared to the respective DMSO controls (FIG. 14B).

Concentrations of OU749: 100 μM for 10% FCS, 200 μM for 10% HS or BSA/HSA 20 mg/ml. Intracellular ROS and superoxide levels (quantified as mean fluorescence intensity [MFI])±standard deviation are depicted. Two-sided t test (*P<0.05; **P<0.01; ***P<0.001).

In light of the above, it is shown that the treatment of MOLM13 and MEC1 with γ-GT inhibitor increases the levels of intracellular reactive oxygen species (ROS) in cell culture systems containing serum (10% FCS and 10% HS) or albumin (BSA and HSA 20 mg/ml).

### Example 14

Treatment of MOLM13 and MEC1 Cells with NF-κB Inhibitors in Combination with γ-G T Inhibitor Increases the Levels of Intracellular Reactive Oxygen Species (ROS) in Cell Culture Systems Containing 10% FCS and 10% HS

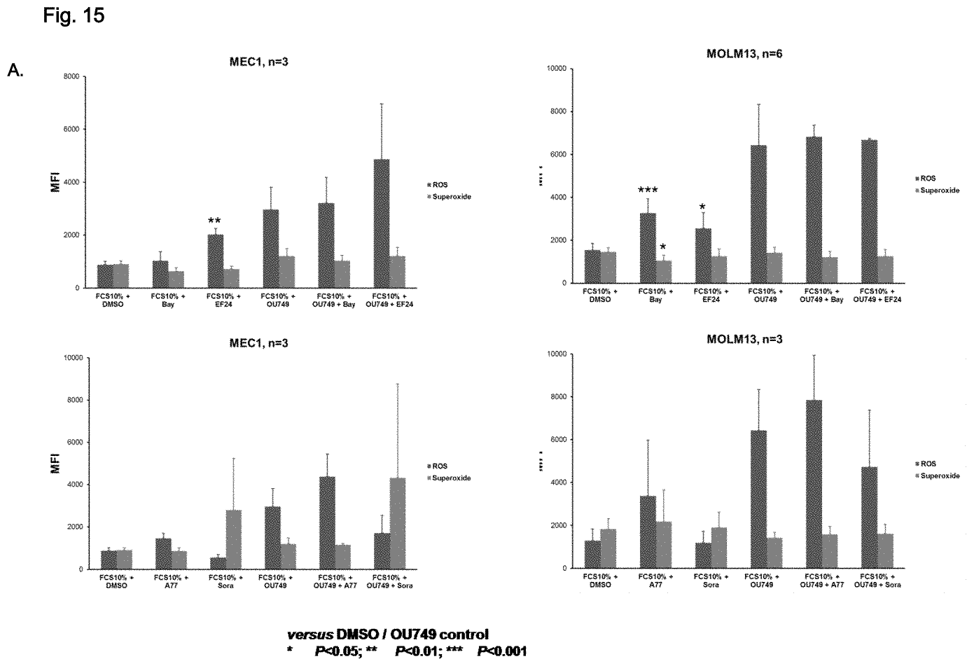

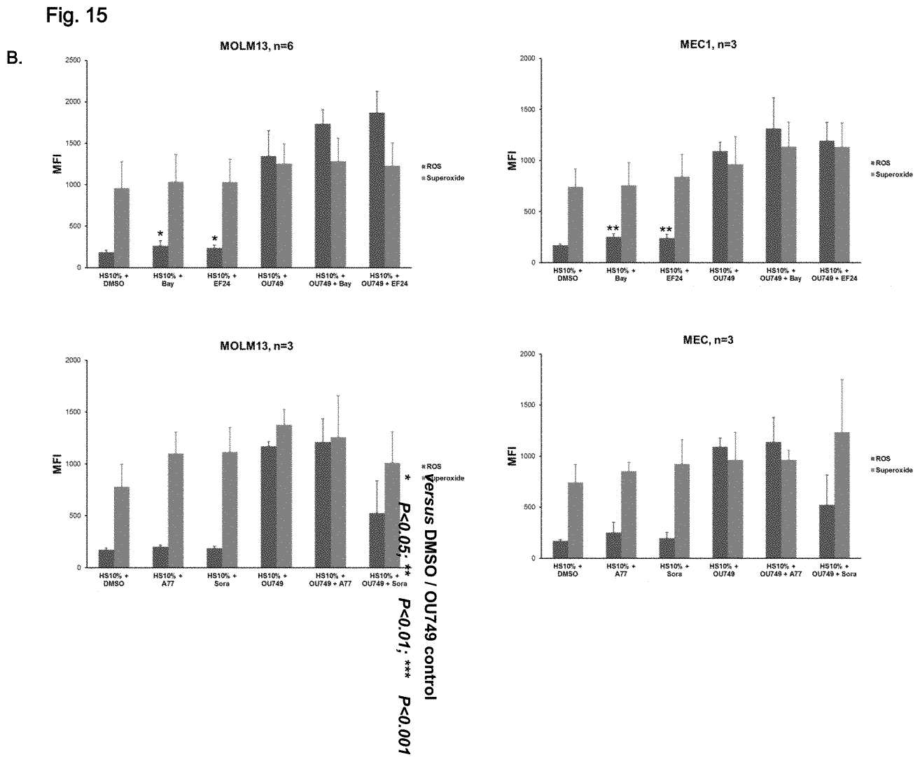

As shown in FIG. 15A, in both MOLM13 and MEC1 cells, treatment with NF-κB inhibitor and γ-GT inhibitor increases intracellular ROS levels in FCS-supplemented cell culture systems.

Similarly, treatment with NF-κB inhibitor and γ-GT inhibitor increases intracellular ROS levels in both HS-supplemented cell culture systems in both MOLM13 and MEC1 cells (FIG. 15B).

Concentrations applied: OU749: 100 μM for 10% FCS, 200 μM for 10% HS. BAY 11-7082: 10 μM, EF24: 5 μM, A77 1726: 100 μM, sorafenib: 10 nM in MOLM13 cells, 10 μM in MEC1 cells.

Intracellular ROS and superoxide levels (quantified as mean fluorescence intensity [MFI])±standard deviation are depicted. Two-sided t test (*P<0.05; **P<0.01).

As such, the treatment of MOLM13 and MEC1 cells with NF-κB inhibitors in combination with γ-GT inhibitor increases the levels of intracellular reactive oxygen species (ROS) in cell culture systems containing 10% FCS and 10% HS.

### Example 15

NF-κB Activity in MOLM13 and MEC1 Cells after 4 h Culture Depends on the Extracellular Environment (i.e. Serum-Free, FCS- or HS-, or HSA-Supplemented Cell Culture Medium)

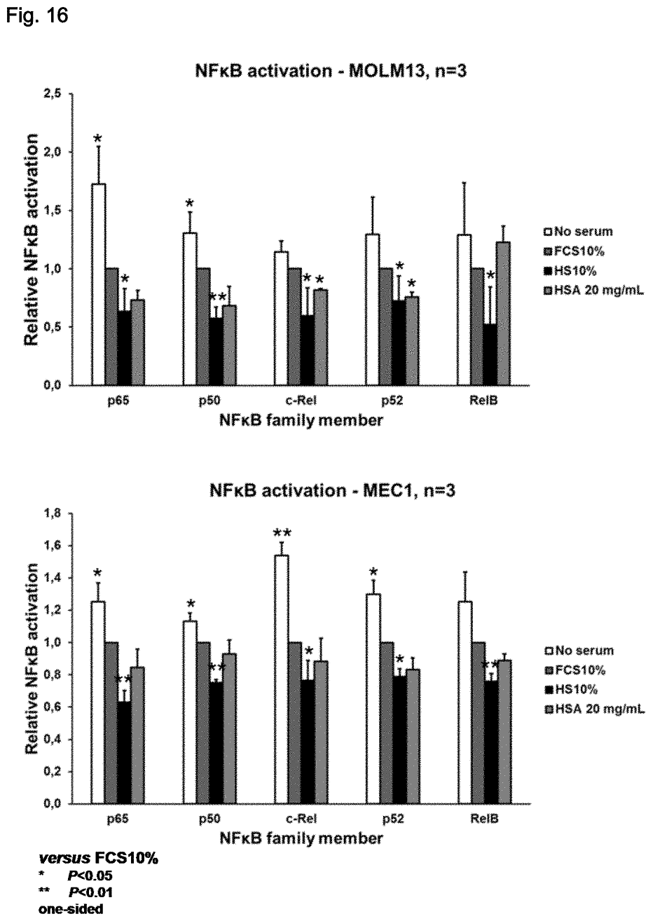

Serum-free culture conditions are associated with enhanced NF-κB activity as shown in FIG. 16. Presence of 10% HS resulted in lower NF-κB activity as compared to FCS-supplemented or serum-free culture conditions, which was reflected by lower nuclear levels of different members of the NF-κB family of transcription factors (p65, p50, c-Rel, p52 and RelB).

Transcription factors levels are given as relative units (relative to levels observed in 10% FCS). One-sided t test (*P<0.05; **P<0.01).

As shown in this Example, the NF-κB activity in MOLM13 and MEC1 cells after 4 h culture depends on the extracellular environment (i.e. serum-free, FCS- or HS-, or HSA-supplemented cell culture medium).

### Example 16

The Inhibitors BAY 11-7082 and EF24 Reduce NF-κB Activity in FCS- but not in HS-Supplemented Cell Culture Medium in MOLM13 and MEC1 after 4 h Treatment and Addition of γ-GT Inhibitor Restores Basal NF-κB Activity in HS-Supplemented Cell Culture Medium and Results in Inhibition of NF-κB Activity Upon Treatment with the Inhibitors BAY 11-7082 and EF24 in HS-Supplemented Cell Culture Medium

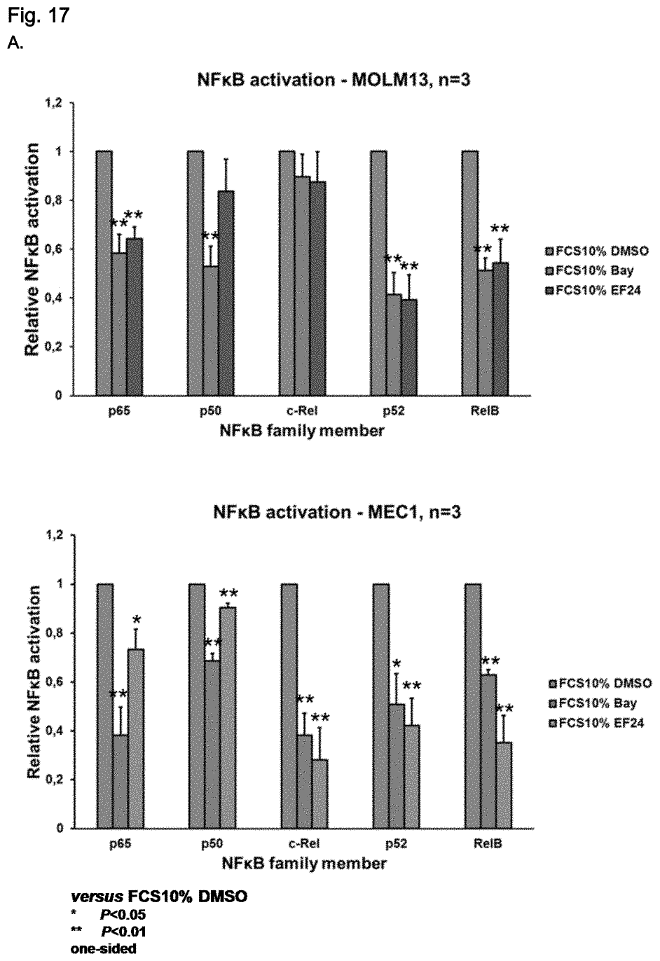

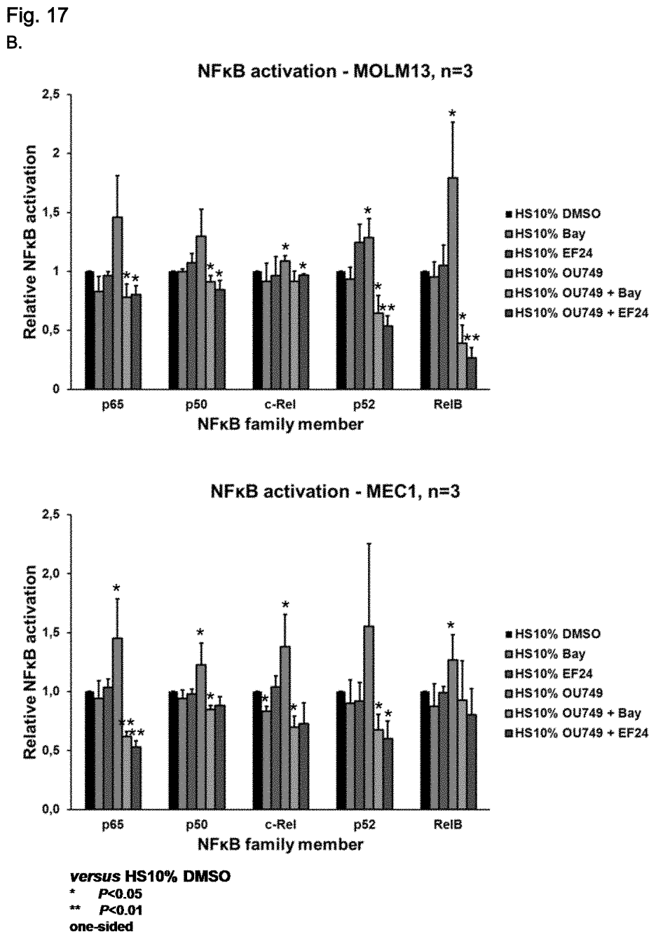

As shown in FIG. 17A, in the presence of 10% FCS, inhibitors of the NF-kB signalling pathway (BAY 11-7082 and EF24) reduce the nuclear levels of different NF-κB transcription factors in both cell lines indicating inhibition of NF-kB transactivation.

In contrast, in the presence of 10% HS no reduction of nuclear levels of NF-κB transcription factors can be observed upon treatment of cells with inhibitors of the NF-kB signalling pathway (BAY 11-7082 and EF24; FIG. 17B). Treatment with 200 μM OU749 results in increased nuclear levels of different members of the NF-κB family of transcription factors in HS-supplemented cell culture medium. Importantly, when a γ-GT inhibitor is present, a decrease in nuclear levels of members of NF-κB family of transcription factors can be observed in HS-supplemented culture systems upon treatment with NF-kB inhibitors indicating inhibition of NF-kB transactivation in the presence of 10% HS.

In FIG. 17, transcription factors levels are given as relative units (relative to levels observed in 10% FCS or 10% HS with DMSO control). Concentrations applied: OU749: 200 μM for 10% HS. BAY 11-7082: 10 μM, EF24: 5 μM. One-sided t test (*P<0.05; **P<0.01).

As such, the NF-κB inhibitors BAY 11-7082 and EF24 only reduce NF-κB activity in FCS- but not in HS-supplemented cell culture medium in MOLM13 and MEC1 cells after 4 h treatment. However, the addition of γ-GT inhibitor in combination with the NF-κB inhibitors BAY 11-7082 and EF24 restores basal NF-κB activity in HS-supplemented cell culture medium and results in inhibition of NF-κB activity upon treatment with the inhibitors BAY 11-7082 and EF24 in HS-supplemented cell culture medium.

### DISCUSSION

### Illustration of the Putative Mechanisms Underlying the Present Invention

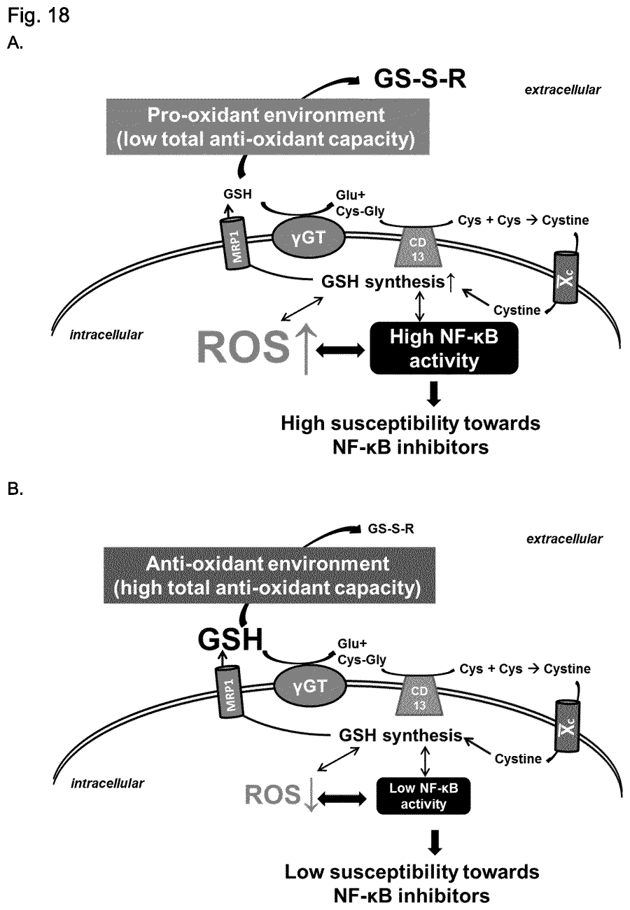

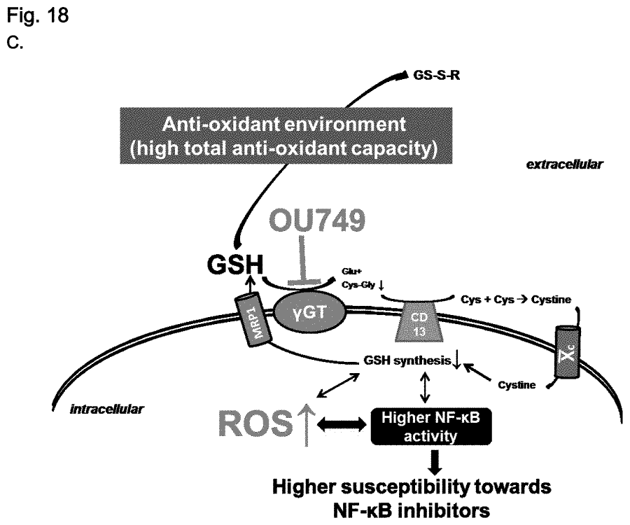

Without wanting to be bound by theory and as shown in the examples above, the present invention is based on the apparent linkage of basal intracellular ROS levels and the anti-oxidant capacity of the extracellular environment. The possible mechanisms underlying the present invention are schematically shown in FIGS. 18A to C.

In particular, as illustrated in FIG. 18A a pro-oxidant extracellular environment (i.e. an extracellular environment with low total anti-oxidative capacity) due to low concentrations of serum, albumins or free thiols, is associated with increased intracellular ROS levels and higher basal activity of the NF-kB signalling pathway. Increased NF-kB signalling makes the cells more susceptible to the action of NF-kB inhibitors. A pro-oxidant environment is possibly generated through increased extracellular oxidation of glutathione, which is known to be exported from cells. Membrane-bound γ-GT appears to play a crucial role in glutathione homeostasis, for example by controlling intracellular synthesis and by contributing to the extracellular export of glutathione. As shown, and without wanting to be bound by theory, higher basal activity of the NF-kB signalling pathway increases the susceptibility/sensitivity of neoplastic cells towards NF-kB inhibitors leading to cell growth inhibition and apoptosis upon treatment with NF-kB inhibitors, i.e. NF-kB inhibitors are more likely to achieve therapeutic efficacy in a “pro-oxidant” environment.

In contrast, and as illustrated in FIG. 18B an anti-oxidant extracellular environment (i.e. an extracellular environment with higher total anti-oxidant capacity) leads to reduced intracellular ROS levels and lower basal activity of the NF-kB signalling pathway—possibly due to due to higher concentrations of serum/albumin and higher free thiol content in the extracellular environment (e.g. in the cell culture system/medium). It is further appears that the lower basal NF-kB signalling activity results in lower susceptibility/sensitivity of neoplastic cells towards NF-kB inhibitors leading to lower efficacy of NF-kB inhibitors in a “anti-oxidant” environment.

In accordance with the present invention the combination of an NF-kB inhibitor and a γ-GT inhibitor achieves efficacy of the NF-kB inhibitor even in and anti-oxidant extracellular environment, i.e. in a cell culture system/medium with high total anti-oxidant capacity due to the presence of serum (in particular human serum), albumins or free thiols, in an anti-oxidant environment. Accordingly, FIG. 18C illustrates that inhibition of γ-GT activity by OU749 increases basal intracellular ROS levels and leads to higher basal NF-kB activity as a consequence.

Without wanting to be bound by theory, this appears to be linked to the putative central role of γ-GT in glutathione homeostasis and is supported by the experimental data of the Examples. As higher basal activity of the NF-kB signalling pathway increases the susceptibility/sensitivity of neoplastic cells towards NF-kB, treatment of neoplastic cells with OU749 may thereby restore/enhance the cells susceptibility/sensitivity to NF-kB. As such, a combination of an NF-kB inhibitor and a γ-GT inhibitor can achieve therapeutic efficacy in neoplastic cells growing in an and high-oxidant environment such as in a cell culture system employing human serum or high albumin or high thiol concentrations as well as in a human patient in situ.

Abbreviations in FIGS. 18A to C: Cys: cysteine; Cys-Gly: cysteinyl-glycine; γ-GT: γ-glutamyltransferase; Glu: glutamate; GSH: glutathione; GS-S-R, oxidized glutathione; ROS: reactive oxygen species; X_{c}: cystine transporter.

### REFERENCES

1. World Health Organization 2005: Geneva, Switzerland. ISBN 92 4 159314 8 [available at: http://www.who.int/cancer/publications/action_against_cancer/en/]
2. Baud & Karin 2009: Nat Rev Drug Discov. 2009; January; 8(1): 33-40. [PubMed: 19116625]
3. Kim et al. 2015: Front Oncol. 2015; 5: 249. [PubMed: 26618142]
4. Vaughan et al. 2016: Oncotarget. 2016; 7(11):12426-46. [PubMed: 26820293]
5. Gilmore 2006: Oncogene. 2006 Oct. 30;25(51):6680-4. [PubMed: 17072321
6. Gilmore & Herscovitch 2006 Oncogene. 2006; 25(51):6887-99. [PubMed: 17072334]
7. Dietrich et al. 2011: Clin Cancer Res. 2012; 18(2):417-31. [PubMed: 22072733]
8. Basseres & Baldwin 2006: Oncogene. 2006 Oct. 30;25(51):6817-30.

[PubMed: 17072330]
9. Bubici et al. 2006: Oncogene. 2006 Oct. 30;25(51):6731-48. [PubMed: 17072325]
10. Turell et al. 2013: Free Radic Biol Med. 2013 December; 65:244-53. [PubMed: 23747983]

1. A composition comprising an NF-κB inhibitor and a γ-glutamyltransferase inhibitor.

2. The composition of claim 1, wherein the γ-glutamyltransferase inhibitor is OU749 and, optionally the NF-κB inhibitor is selected from the group consisting of: BAY 11-7082; EF24; leflunomide; sorafenib; bortezomib; thalidomide; lenalidomide; arsenic trioxide; and ibrutinib.

3. A method of treating a subject having a malignant disease comprising administering a therapeutically effective amount of the composition of claim 1 to a subject having a malignant disease, preferably caused by TP53-mutated cells, preferably the disease is selected from the group consisting of: ovarian cancers; colorectal cancers; oesophageal cancers; head and neck cancers; laryngeal cancers; lung cancers; skin cancers; pancreatic cancers; stomach cancers; liver cancers; brain cancers; bladder cancers; breast cancers; uterus cancers; soft tissue cancers; leukaemias; lymphomas; prostate cancer; bone cancers; endocrine gland cancers; testicle cancers; kidney cancers; haematopoietic cancers; cervical cancers; cholangiocarcinoma; Li-Fraumeni syndrome; osteosarcoma; rhabdomyocarcinoma; adrenocortical carcinoma; chronic lymphatic leukaemia (CLL); myelodysplastic syndromes (MDS); and acute myeloid leukaemia (AML).

4. The method of claim 3, wherein the TP53-mutated cells are refractory to a chemotherapeutic agent, and optionally the disease is a chronic disease, preferably the disease is chronic lymphatic leukaemia (CLL), and optionally the disease is an acute disease, preferably the disease is acute myeloid leukaemia (AML).

5-7. (canceled)

8. The composition of claim 1, comprising between 4 and 1000 μM of the NF-κB inhibitor and between 3 and 1000 μM of the γ-glutamyltransferase inhibitor.

9. An in vitro method of identifying an NF-κB inhibitor for its potential to act as a therapeutically effective NF-κB inhibitor in the treatment of a malignant disease, preferably caused by TP53-mutated cells, in vivo, said method comprising exposing an in vitro culture of malignant human cells comprising human serum to the NF-κB inhibitor and to a γ-glutamyltransferase inhibitor, wherein the NF-κB inhibitor is identified as having high potential to act as a therapeutically effective NF-κB inhibitor in the treatment of a malignant disease, preferably caused by TP53-mutated cells, in vivo, if a cytostatic and/or cytotoxic effect on the cells is observable.

10. The method of claim 9, wherein the method comprises the steps of:
(a) exposing an in vitro culture of malignant human cells to the NF-κB inhibitor and assessing the cytostatic and/or cytotoxic effect on the cells, wherein the culture does not contain human serum;
(b) exposing an in vitro culture of malignant human cells to the NF-κB inhibitor and assessing the cytostatic and/or cytotoxic effect on the cells, wherein the culture contains human serum;
(c) exposing an in vitro culture of malignant human cells corresponding to the culture of (b) to the NF-κB inhibitor and to the γ-glutamyltransferase inhibitor and assessing the cytostatic and/or cytotoxic effect on the cells; and
(d) comparing the cytostatic and/or cytotoxic effects assessed in (a), (b) and (c), wherein when the NF-κB inhibitor's cytostatic and/or cytotoxic effect is decreased or unchanged in (b) compared to (a) but is restored or enhanced in (c) the NF-κB inhibitor is identified as having high potential to act as a therapeutically effective NF-κB inhibitor in the treatment of a malignant disease, preferably caused by TP53-mutated cells, in vivo.

11. (canceled)

12. The method of claim 9, wherein the γ-glutamyltransferase inhibitor is OU749 and/or the NF-κB inhibitor is selected from the group consisting of: BAY 11-7082; EF24; leflunomide; sorafenib; bortezomib; thalidomide; lenalidomide; arsenic trioxide; and ibrutinib, optionally the cytostatic effect is assessed by assessing cell proliferation, optionally the cytotoxic effect is assessed by assessing induction of apoptosis.

13. A kit comprising a γ-glutamyltransferase inhibitor and an NF-κB inhibitor for use in the treatment of a malignant disease, preferably caused by TP53-mutated cells, in a human patient.

14. The kit of claim 13, wherein the γ-glutamyltransferase inhibitor is OU749 and/or the NF-κB inhibitor is selected from the group consisting of: BAY 11-7082; EF24; leflunomide; sorafenib; bortezomib; thalidomide; lenalidomide; arsenic trioxide; and ibrutinib, preferably the disease is selected from the group consisting of: ovarian cancers; colorectal cancers; oesophageal cancers; head and neck cancers; laryngeal cancers; lung cancers; skin cancers; pancreatic cancers; stomach cancers; liver cancers; brain cancers; bladder cancers; breast cancers; uterus cancers; soft tissue cancers; leukaemias; lymphomas; prostate cancer; bone cancers; endocrine gland cancers; testicle cancers; kidney cancers; haematopoietic cancers; cervical cancers; cholangiocarcinoma; Li-Fraumeni syndrome; osteosarcoma; rhabdomyocarcinoma; adrenocortical carcinoma; chronic lymphatic leukaemia (CLL); myelodysplastic syndromes (MDS) and acute myeloid leukaemia (AML).

15. A method for increasing efficacy of an NF-κB inhibitor against TP53-mutated cells in vitro comprising exposing TP53-mutated cells in vitro to an NF-κB inhibitor and a γ-glutamyltransferase inhibitor.

16. A method of treating a subject having a malignant disease comprising administering a therapeutically effective amount of the composition of claim 2 to a subject having a malignant disease, preferably caused by TP53-mutated cells, preferably the disease is selected from the group consisting of: ovarian cancers; colorectal cancers; oesophageal cancers; head and neck cancers; laryngeal cancers; lung cancers; skin cancers; pancreatic cancers; stomach cancers; liver cancers; brain cancers; bladder cancers; breast cancers; uterus cancers; soft tissue cancers; leukaemias; lymphomas; prostate cancer; bone cancers; endocrine gland cancers; testicle cancers; kidney cancers; haematopoietic cancers; cervical cancers; cholangiocarcinoma; Li-Fraumeni syndrome; osteosarcoma; rhabdomyocarcinoma; adrenocortical carcinoma; chronic lymphatic leukaemia (CLL); myelodysplastic syndromes (MDS); and acute myeloid leukaemia (AML).

17. The method of claim 16, wherein the TP53-mutated cells are refractory to a chemotherapeutic agent, and optionally the disease is a chronic disease, preferably the disease is chronic lymphatic leukaemia (CLL), and optionally the disease is an acute disease, preferably the disease is acute myeloid leukaemia (AML).

18. The method of claim 3, wherein the composition comprises between 4 and 1000 μM of the NF-κB inhibitor and between 3 and 1000 μM of the γ-glutamyltransferase inhibitor.

19. The method of claim 10, wherein the γ-glutamyltransferase inhibitor is OU749 and/or the NF-κB inhibitor is selected from the group consisting of: BAY 11-7082; EF24; leflunomide; sorafenib; bortezomib; thalidomide; lenalidomide; arsenic trioxide; and ibrutinib, optionally the cytostatic effect is assessed by assessing cell proliferation, optionally the cytotoxic effect is assessed by assessing induction of apoptosis.
